(19) United States
(12) Patent Application Publication
(10) Pub. No.: US 2015/0005827 A1
(43) Pub. Date: Jan. 1, 2015

(54) METHOD FOR OPERATING ON A SPINE BY USING A SPINAL FIXATION DEVICE

(71) Applicant: KWAN KU LIN, Pasadena, CA (US)

(72) Inventor: CHIH I LIN, (US)

(73) Assignee: SPIRIT SPINE HOLDING CORPORATION, INC., Pasadena, CA (US)

(21) Appl. No.: 14/488,764

(22) Filed: Sep. 17, 2014

Related U.S. Application Data
(62) Division of application No. 12/945,983, filed on Nov. 15, 2010.

(51) Int. Cl. A61B 17/70

(52) U.S. Cl. CPC A61B 17/7098; A61B 17/7032; A61B 2017/564
USPC 606/279

(57) ABSTRACT

This invention relates to a method for operating on a spine, and more particularly to a method for operating on a spine by using a spinal fixation device which comprises the steps of (1) creating a hole on a vertebra; (2) placing said hollow fixation part joined with said cover part into the hole, and leaving the joining end of said hollow fixation part outside of the vertebra and exposed; (3) injecting said medical filling into said cover part via an injecting device and said hollow fixation part; (4) allowing said medical filling to become solidified so that said medical filling and the cover part form a lump; (5) removing said injecting device from said hollow fixation part.

### CROSS-REFERENCE TO RELATED APPLICATION

This Application is a divisional application of co-pending application Ser. No. 12/945,983 filed on Nov. 15, 2010 by Chih I Lin.

### FIELD OF THE INVENTION

The invention relates to a method for operating on a spine, and more particularly to a method for operating on a spine by using a spinal fixation device.

### DESCRIPTION OF PRIOR ART

Traditionally, bone fixation devices are designed differently according to various purposes, as exemplified by the bone nails found in spine fixation devices (refer to Taiwan Patent No. 250680, Component No. 20), the inner bone nails used in long bone fixation devices (refer to Taiwan Patent No. M316059, Component No. 3), as well as the screws used in artificial hip joints (please refer to Taiwan Patent No. 153907 and 245118, Component No. 30), all of which arc structurally different from each other. However, none of said bone nails found in spine fixation devices, said inner bone nails found in long bone fixation devices, or said screws used in artificial hip joints can be integrally combined with the cancellous bones from the sites awaiting surgical fixation. For instance, for the bone nails found in spine fixation devices, the bone nails are inserted and secured into a vertebra simply by allowing the spaces between the crests on each nail to fit into the external cortical bone and the internal cancellous hone of said vertebra. Therefore, the bone nails could become loosened from the surgical sites due to losses of bone mass for the bone nails to hold onto, caused by changes in affected patients' weight, activities, bone growth, severe osteoporosis (which is the loss of cancellous bone), or other factors.

In order to resolve the aforesaid shortcomings, a number of patents had modified the bone nails structurally to enhance the stability thereof. For example, in the patent EP0714643, the bone nails have threads disposed thereon, so as to increase the contact area between the bone nails and the bones; in the patent DE19801219, hardened barb-like protrusions are arranged surroundingly on a perimeter of each bone nail, so that the barb-like protrusions can secure the bone nails against loosening. Similar examples can also be found in Taiwan Patent No. 200716052, which also utilizes barbs to prevent the bone nails from loosening from bones; as well as in Taiwan Patent No. M306498, which applies the concept of expansive screws on the bone nails, so that the bone nails can be secured in bones more effectively and firmly. Overall, the aforesaid patents aimed to reduce the incidence of bone nails loosened from bones after surgery by structurally enhancing the bone nails. However, said incidents still occur and do greater damage to the affected patients, due to the fact that most of the bone nails are made of metal, which are structurally stronger than bones, and often leads to loosening of bone nails from bones after the bones become worn off by the nails.

In known prior arts, bone cement and bone nails are used in combination on patients suffering from osteoporosis, in which bone cement is injected into a patient's bone first, and then a bone nail is screwed through a bone plate and into the bone injected with the bone cement. As the bone nail is screwed into the bone, spaces between the superficial threads of the bone nail and the bone are filled up by the bone cement, so as to further secure the bone nail within the bone when the bone cement becomes solidified. But the solidification of bone cement could also prevent the body of bone nail from completely entering into the bone, or conversely becomes damaged when hone nail is entered, which would result in the loosening of bone nail and bone plate in both cases.

In Taiwan Patents M306498, M346406, 267342, or other similar patents, a medical filling such as bone cement is injected via an opening disposed on a hollow bone nail, wherein the medical filling is usually a bone cement being made into a pulp-like form before surgery, and becomes solidified after surgery, so as to stabilize the bone nail after being injected into surgical sites. But in actual applications, the injected bone cement often accumulates or even forms spikes around small openings due to high viscosity thereof. As a result, the affected surgical site cannot grow more bone mass for the bone nail to hold onto, which restricts the stability of the hone nail consequently.

In the bone fixation device of the present invention, a hollow fixation part has a cover part joined therewith, which may be used to limit the scope for injecting a medical filling, such that the medical filling is allowed to form a lump-like structure with said cover part after being injected; said lump-like structure then further fixes the bone fixation device of this invention onto a surgical site awaiting fixation (such as a vertebra, a hip joint, or a long bone).

### SUMMARY OF THE INVENTION

A primary objective of the invention is to provide a bone fixation device.

Another objective of the invention is to provide a bone fixation device having a cover part, and being injectable with a medical filling via a hollow fixation part thereof.

Yet another objective of the invention is to provide a bone fixation device in which said cover part may be expanded after a medical filling is injected thereinto.

Yet another objective of the invention is to provide a bone fixation device in which said medical filling may be injected into a cover part thereof, and subsequently becomes solidified and secured on bones.

Still another objective of the invention is to provide a bone fixation device having a cover part that may limit the scope for injecting a medical filling.

Yet another objective of the invention is to provide a bone fixation device having a cover part that may form a lump-like structure with a medical filling, after the medical filling is injected.

A further objective of the invention is to provide a spine fixation system using bone joining devices to join with bone fixation devices.

Yet another objective of the invention is to provide an artificial hip joint replacement system using hip joint substitutes and acetabular implants to join with bone fixation devices.

Still another objective of the invention is to provide a long bone fixation system using tightening devices to join with a plurality of bone fixation devices.

The bone fixation device of the present invention, comprising:
a hollow fixation part having a joining end at a proximal end thereof, and an injecting end at a distal end thereof;
a cover part being joined with the injecting end of said hollow fixation part;
a medical filling being forceable and/or injectable via the injecting end of said hollow fixation part and into said cover part, such that the medical filling is allowed to become lump-like after injecting.

The joining end of said hollow fixation part, which can be used to join with any known bone joining devices (such as the joining rods or the bone plates used for spine fixation devices, and the acetabular implants used for artificial hip joint replacement devices), or any known tightening devices (such as the nuts used for long bone fixation devices), such that after injecting a medical filling, said bone fixation device secures bones of surgical sites to the expanded and solidified cover part, as well as fitting said bones to the bone joining device at the joining end of said hollow fixation part, thereby enhancing the stability of said bone fixation device.

The joining end of said hollow fixation part, which may further comprise a tightening mechanism when joined with known bone joining devices. When joined with the bone joining devices, said tightening mechanism may be used to adjust distances between the bone joining devices and lump-like medical fillings, so as to strengthen the stability of said bone fixation devices.

The joining end of said hollow fixation part, which can be joined with bone joining devices (including tightening mechanisms if necessary) or tightening devices by using any known methods of joining, such as by the formation into a unibody (refer to FIGS. 3b, 3c, 3d, and 3e), locking, clasping, or screwing.

In said hollow fixation part, a body between the joining end and the injecting end may include various textures like: annular textures, striped textures, speckled textures, or mesh-like textures (refer to FIGS. 3b, 3c, and 3d); so as to increase the friction between said hollow fixation part and bones, thus preventing said hollow fixation part from loosening after surgery.

The injecting end of said hollow fixation part, which may be of any known configurations, such as an injecting end having multiple slits or holes (refer to FIGS. 5b and 5c), and is more preferably configured with multiple slits.

Said cover part may be secured to the body, the injecting end, or the joining end of said hollow fixation part, and is more preferably secured to the body.

Said hollow fixation part and said cover part may be joined together by using any known methods of joining. As the body of said hollow fixation part has an annular trough disposed thereon, and the annular trough is used to secure said cover part to the body of said hollow fixation part, wherein the cover part may be secured to the annular trough by using any known methods of joining, such as by riveting, clasping, locking, adhering, or binding (refer to FIGS. 8a, 8b, 8c, and 8d), and is more preferably secured by riveting or clasping.

Said cover part may completely or partially cover the injecting end of said hollow fixation part.

Said cover part may be of any known cover parts, such as elastic cover parts, mesh-like cover parts, porous cover parts, or metal cover parts (refer to FIGS. 7a, 7b, 7c, and 7d).

Said cover part may be of any shapes, such as cylindrical, cone-like, ball-like, quasi-balls, or quasi-cubes.

Said medical filling is allowed to become lump-like after injecting, wherein said lump-like structure may be of any known shapes, such as cylindrical, cone-like, ball-like, quasi-balls, or quasi-cubes, and is more preferably cylindrical.

Said medical filling may be any known medical fillings, such as bone grafts, bone substitutes, bone cement, and/or a mixture, a composition, a composite thereof, as referred to in TW-097141700, TW-1227146, and U.S.-20070088436. Generally, said medical filling could be as simple as bone grafts like autografts or allografts, but this result in relatively less secure fixation of bones. Therefore, said medical filling is more preferably to include at least a pulp that can become solidified, such as a pulp-like medical filling, or be filled with said bone grafts and then injected with a solidifiable pulp, such that the pulp is allowed to completely fill up or even slightly expand the lump-like structure, so as to strengthen joining thereof. Said medical filling is most preferably to be a pulp-like medical filling. The pulp-like medical filling refers to a medical filling that can be made into a pulp during or before actual use, and become solidified within an adequate amount of time after being injecting into the lump-like structure. For example, this may include the aforesaid bone substitutes, bone cement, and/or a mixture, a composition, a composite thereof. Said medical filling is preferably to be osteo-conductive and/or osteo-inductive, such as the known FIA type of bone fillings, and is more preferably to be osteo-inductive like the SrHA type of medical fillings, as referred to in TW-097141700. Anyone of ordinary skill in the art knows that in addition to the known medical fillings, the above-described medical filling may be substituted with any medical fillings reconstituted or modified from any known medical fillings, or with any newly developed medical fillings having equivalent effects.

Said hollow fixation part may be made of a single section or a combination of multiple sections. A single-section hollow fixation part is hollow throughout the section; whereas a multiple-section fixation part has a hollow distal injecting end, and other ends may be hollow or solid (refer to FIG. 2b). Each of the sections may be joined together by using any known methods of joining, such as by locking, clasping, or screwing (refer to FIGS. 6b and 6c).

Said bone fixation device may be used in combination with a bone joining device, in order to make up a spine fixation system (refer to FIG. 15), which is the spine fixation system of the present invention, comprising:
a plurality of fixation parts having joining ends at proximal ends thereof, and distal ends thereof are used to secure vertebrae awaiting fixation;
a bone joining device joined with the joining end of said fixation part; characterized in that:

Said fixation part includes at least one hollow fixation part having a joining end at a proximal end thereof, and an injecting end at a distal end thereof; at least one said hollow fixation part being further joined with a cover part, so as to force or inject a medical filling via the injecting end of said hollow fixation part and into said cover part, such that the medical filling is allowed to become lump-like after injecting.

Said fixation part may be any known fixation parts, such as bone nails, screws, and/or hollow fixation parts, as referred to in the following diagrams and descriptions thereof.

Said bone joining device may be any known bone joining devices, which may be a single part like a bone plate, or made up of a plurality of parts like joining rods, tightening devices, receiving troughs, rods, or barbs (refer to FIGS. 4a, 4b, 4c, 4d, and 4e).

The above-described hollow fixation part, cover part, and medical filling are as described before.

The present invention also discloses a method for operating on a spine, comprising the steps of:
(1) creating a hole on a vertebra;
(2) placing said hollow fixation part and said cover part joined therewith into the hole, and leaving the joining end of said hollow fixation part outside of the vertebra and exposed;
(3) injecting said medical filling into said cover part via an injecting device and said hollow fixation part;
(4) injecting said medical filling so that said medical filling and the cover part together form a lump;
(5) removing said injecting device from said hollow fixation part.

Said step (1) may utilize any known steps for creating holes, an example of which can be referred to in TW-557785.

Said step (2) may use any known steps for placing, such as using a tube to place said hollow fixation part and said cover part joined therewith into the hole, as indicated in FIG. 10a, or directly placing the spine fixation system into the hole, as indicated in FIG. 12a.

Preferably, said injecting device is joined with the joining end of said hollow fixation part, as shown in FIG. 10a.

Said steps (3) and (4) may use any known steps for injecting, characterized in that the cover part is used to limit a scope for injecting the medical filling.

Said step (5) may use any known steps for removing injecting devices.

The method of this invention may further comprise a step (6), in which a bone joining device is joined with the joining end of said hollow fixation part.

Said bone fixation device may be used in combination with a hip joint substitute and an acetabular implant to form an artificial hip joint replacement system (refer to FIGS. 16a, 16b, 17, 18, 19, 20, 21, 22a, and 22b), which is the artificial hip joint replacement system of this invention, comprising:
a hollow fixation part having a joining end at a proximal end thereof, and an injecting end at a distal end thereof;
a cover part being joined with the injecting end of said hollow fixation part;
a hip joint substitute being joined with the hollow fixation part, and is used for covering over a pre-finished femur;
an acetabular implant being joined with the joining end of said hollow fixation part; and
a medical filling being forceable and/or injectable via the injecting end of said hollow fixation part and into said cover part, such that the medical filling is allowed to become lump-like after injecting.

Said hollow fixation part allows said artificial hip joint replacement system to secure bones of surgical sites to the expanded and solidified cover part after injecting the medical filling, and the joining of said hollow fixation part with a tightening device further stabilizes said artificial hip joint replacement system.

Said hollow fixation part can be joined with hip joint substitutes by using any known methods of joining, such as by the formation into a unibody (refer to FIG. 16a), adhering, locking, clasping, or screwing.

Said cover part may further include a halting part for allowing a guiding device to go through the injecting end of said hollow fixation part. Said guiding device pushes against the halting part such that said cover part is guided into an injecting position when inserted into a bone, and is not squeezed or collapsed.

Said hip joint substitute may be any known hip joint substitutes, like the ones described in the Taiwan Patent No. 245118 and 153907.

Said hip joint substitute may he joined with tightening devices by using any known methods of joining, such as by locking, clasping, or screwing.

Said acetabular implant may be any known acetabular implants, like the ones described in the Taiwan Patent No. 245118 and 153907.

Said artificial hip joint replacement system may further include a plurality of hollow fixation parts and cover parts (refer to FIG. 22a), which are also injected with a medical filling so as to strengthen the stability of said artificial hip joint replacement system.

Said artificial hip joint replacement system may further include a supplementary fixation device for further strengthening the stability thereof, which can be a securing cable, a bone plate, a joining rod, a bone nail, a screw, a rod, or a barb.

Said hip joint replacement system may be joined with the supplementary fixation device by using any known methods of joining, such as by locking, clasping, or screwing.

The above-described hollow fixation part, cover part, and medical filling are as described before.

Said bone fixation device may also be used in combination with a tightening device to form a long bone fixation system (refer to FIGS. 23-28 and 29a-29k), which is the long bone fixation system of the present invention, comprising:
a hollow fixation part having a joining end at a proximal end thereof, and an injecting end at a distal end thereof;
a cover part being joined with the injecting end of said hollow fixation part;
a medical filling being forceable and/or injectable via the injecting end of said hollow fixation part and into said cover part, such that the medical filling is allowed to become lump-like after injecting; and
a tightening device being joined with the joining end of said hollow fixation part, so as to tighten the lump-like cover part against the bones of surgical sites.

Said tightening device may be any known tightening devices, as long as it can be used to tighten the lump-like cover part against the bones of surgical sites and strengthen the stability of said long bone fixation system, such as screws and bone plates/screws (refer to FIG. 25).

The joining end of said hollow fixation part may be used to join with any known tightening devices, such that said long bone fixation system secures the bones of surgical sites to the expanded and solidified cover part, and the bone joining device fitted on the joining end of said hollow fixation part after injecting the medical filling, so as to enhance the stability of said long bone fixation system.

The joining end of said hollow fixation part may be joined with the tightening devices by using any known methods of joining, such as by locking, clasping, or screwing.

Said cover part may further include a halting part for allowing a guiding device to go through the injecting end of said hollow fixation part. Said guiding device pushes against the halting part so that said cover part is guided into an injecting position when inserted into a bone, and is not squeezed or collapsed.

Said hollow fixation part may further include a second cover part, which can be joined with the body of the hollow fixation part, thus further strengthening the coupling between two broken bones (refer to FIG. 28).

Said long bone fixation system may further include a supplementary fixation device for further strengthening the stability thereof, which can be a bone plate, a joining rod, a bone nail, a screw, a rod, or a barb.

### BRIEF DESCRIPTION OF DRAWINGS

FIG. 1a is a schematic view showing a fully assembled bone fixation device according to a preferred embodiment of the present invention.

FIG. 1b is a schematic view showing the bone fixation device of FIG. 1a after injecting is completed.

FIG. 2a is a schematic view showing a fully disassembled bone fixation device according to a preferred embodiment of the present invention.

FIG. 2b is a schematic view showing a fully disassembled bone fixation device according to another preferred embodiment of the present invention.

FIGS. 3a, 3b, 3c, 3d, and 3e are schematic views showing five different types of body texture for a hollow fixation part of the bone fixation device of the present invention.

FIGS. 4a, 4b, 4c, 4d, and 4e are schematic views showing the joining between the hollow fixation device of the bone fixation device with four types of bone joining device according to a preferred embodiment of the present invention.

FIGS. 5a, 5b, and 5c are schematic views showing three types of injecting end of the hollow fixation device of the bone fixation device according to a preferred embodiment of the present invention.

FIGS. 6a, 6b, and 6c are schematic views showing three types of coupling for the hollow fixation device of the bone fixation device according to a preferred embodiment of the present invention.

FIGS. 7a, 7b, 7c, and 7d are schematic views showing four types of cover part for the bone fixation device according to a preferred embodiment of the present invention.

FIGS. 8a, 8b, 8c, and 8d are schematic views showing the joining between four types of cover part with the hollow fixation part of the bone fixation device according to a preferred embodiment of the present invention.

FIGS. 9a, 9b, and 9c are schematic views showing the joining between three types of metal cover part with the hollow fixation device of the bone fixation device according to a preferred embodiment of the present invention.

FIGS. 10a, 10b, 10c, 10d, 10e, and 10f are schematic views showing the surgical steps for joining the bone fixation device of the invention with a bone joining device.

FIGS. 11a, 11b, and 11c are schematic views showing the joining between the bone fixation device of the invention with a bone joining device having a tightening mechanism.

FIGS. 12a, 12b, and 12c are schematic views showing the surgical steps for joining the bone fixation device of the invention with a cover part made of PET.

FIGS. 13a, 13b, 13c, 13d, and 13e are schematic views showing the surgical steps for joining the bone fixation device of the invention with a bone joining device having a tightening mechanism.

FIGS. 14a, 14b, and 14c are schematic views showing the surgical steps for joining the bone fixation device of the invention with an elastic metal cover part.

FIG. 15 is a schematic view showing a spine fixation system comprised of the bone fixation device of the invention in combination with a bone joining device.

FIG. 16a is a schematic view showing a hip joint replacement system comprising the bone fixation device being joined with a hip joint substitute and an acetabular implant, according to a preferred embodiment of the present invention.

FIG. 16b is a schematic view showing the system of FIG. 16a after injecting is completed according to a preferred embodiment of the present invention.

FIG. 17 is a schematic view showing an artificial hip joint replacement system comprising the bone fixation device being joined with a hip joint substitute and an acetabular implant, according to another preferred embodiment of the present invention.

FIG. 18 is a schematic view showing an artificial hip joint replacement system comprising the bone fixation device being joined with a hip joint substitute and an acetabular implant, according to yet another preferred embodiment of the present invention.

FIG. 19 is a schematic view showing an artificial hip joint replacement system comprising the bone fixation device being joined with a hip joint substitute and an acetabular implant, according to still another preferred embodiment of the present invention.

FIG. 20 is a schematic view showing an artificial hip joint replacement system comprising the bone fixation device being joined with a hip joint substitute and an acetabular implant, according to a further preferred embodiment of the present invention.

FIG. 21 is a schematic view showing an artificial hip joint replacement system comprising the bone fixation device being joined with a hip joint substitute and an acetabular implant, according to yet another preferred embodiment of the present invention.

FIG. 22a is a schematic view showing an artificial hip joint replacement system comprising the bone fixation device being joined with a hip joint substitute and an acetabular implant, according to still another preferred embodiment of the present invention.

FIG. 22b is a schematic view showing the system of FIG. 22a from another angle of view.

FIG. 23 is a schematic view showing a long bone fixation system comprising the bone fixation device being joined with a tightening device, according to a preferred embodiment of the present invention.

FIG. 24 is a schematic view showing a long bone fixation system comprising the bone fixation device being joined with a tightening device, according to another preferred embodiment of the present invention.

FIG. 25 is a schematic view showing a long bone fixation system comprising the bone fixation device being joined with a tightening device, according to yet another preferred embodiment of the present invention.

FIG. 26 is a schematic view showing a long bone fixation system comprising the bone fixation device being joined with a tightening device, according to yet another preferred embodiment of the present invention.

FIG. 27 is a schematic view showing a long bone fixation system comprising the bone fixation device being joined with a tightening device, according to still another preferred embodiment of the present invention.

FIG. 28 is a schematic view showing a long bone fixation system comprising the bone fixation device being joined with a tightening device, according to a further preferred embodiment of the present invention.

FIGS. 29a to 29k are schematic views showing the surgical steps for installing the long bone fixation system of the invention.

### DETAILED DESCRIPTION OF PREFERRED EMBODIMENTS

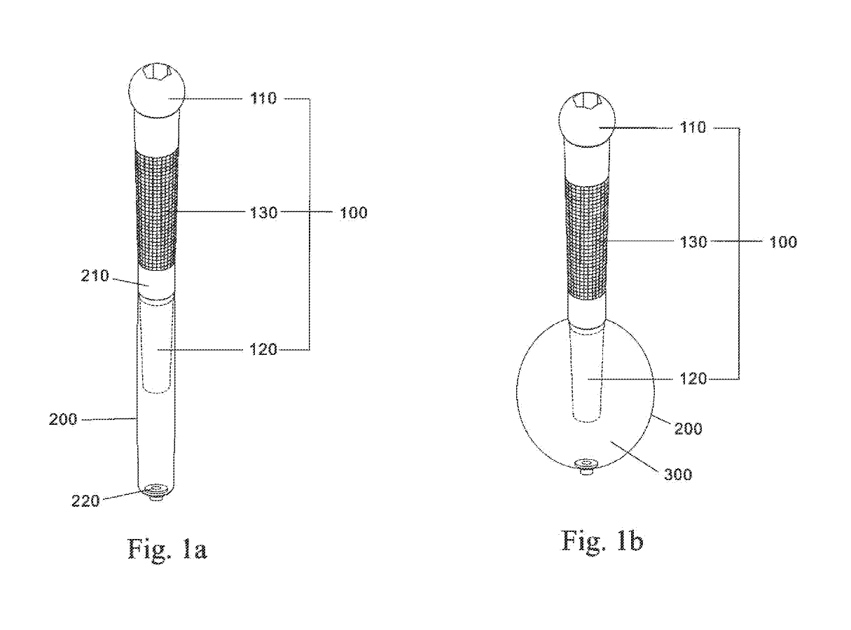

FIG. 1a is a schematic view showing a fully assembled bone fixation device according to a preferred embodiment of the present invention. In which 100 is a hollow fixation part, 110 is a joining end, 120 is an injecting end, 130 is a body having a mesh-like texture superficially, 200 is a cover part, 210 is a securing ring, and 220 is a halting part. The hollow fixation part 100 is formed as a unibody, and the securing ring 210 is used to secure the cover part 200 to the body 130 of the hollow fixation part 100.

FIG. 1b is a schematic view showing the bone fixation device of FIG. 1a after injecting is completed. In which 100 is a hollow fixation part, 110 is a joining end, 120 is an injecting end, 130 is a body, 200 is a cover part, 210 is a securing ring, 220 is a halting part, and 300 is a medical filling. Wherein the medical filling 300 is injected into the cover part 200 via the injecting end 120 of the hollow fixation part 100, and the medical filling 300 is allowed to completely fill up the cover part 200 and become lump-like.

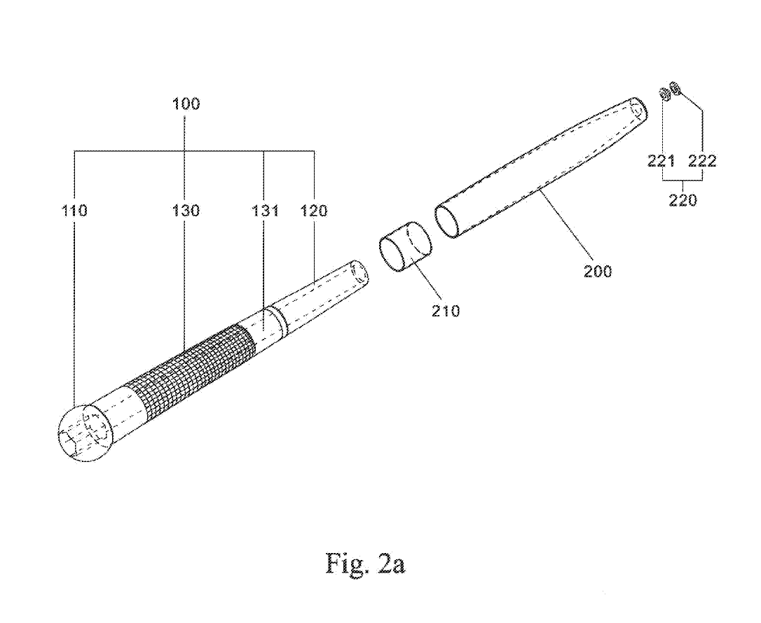

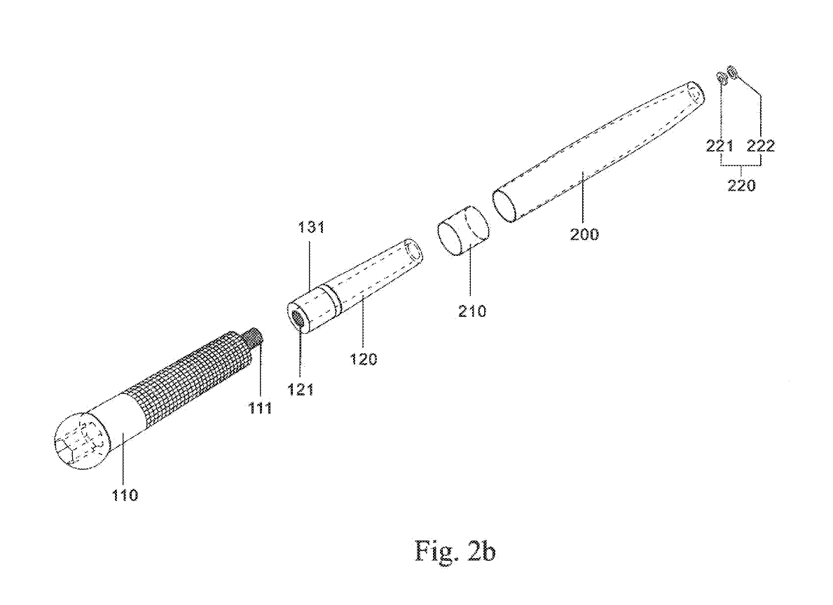

FIG. 2a is a schematic view showing a fully disassembled bone fixation device according to a preferred embodiment of the present invention. In which 100 is a hollow fixation part, 110 is a joining end, 120 is an injecting end, 130 is a body, 131 is an annular trough, 200 is a cover part, 210 is a securing ring, 220 is a halting part, 221 is an anterior halting component, and 222 is a posterior halting component. Moreover, the securing ring 210 is used to secure the cover part 200 to the annular trough 131 of the hollow fixation part 100.

FIG. 2b is a schematic view showing a fully disassembled bone fixation device according to another preferred embodiment of the present invention. In which 110 is a joining end, 111 is a coupling end of the joining end 110, 120 is an injecting end, 121 is a coupling end of the injecting end 120, 131 is an annular trough, 200 is a cover part, 210 is a securing ring, 220 is a halting part, 221 is an anterior halting component, and 222 is a posterior halting component. Wherein the hollow fixation part is divided into two portions including the joining end 110 and the injecting end 120, and the joining end 110 and the injecting end 120 are joined together by screwing. Moreover, the securing ring 210 is used to secure the cover part 200 to the annular trough 131 of the hollow fixation part 100.

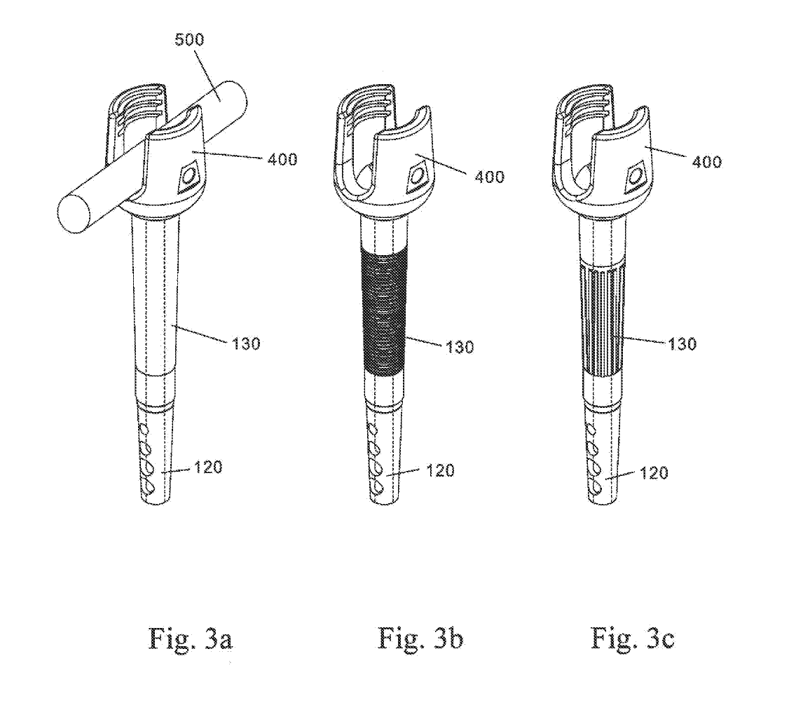

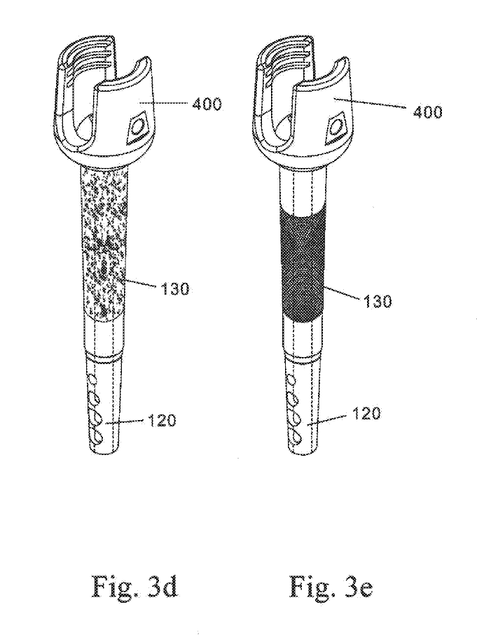

FIGS. 3a, 3b, 3c, 3d, and 3e are schematic views showing five different types of body texture for a hollow fixation part of the bone fixation device of the present invention. FIG. 3a shows: A bone joining device 400 and a hollow fixation part are formed as a unibody, a body 130 having no textures, an injecting end 120 having a plurality of holes for injecting a medical filling into a cover part via the injecting end 120. FIG. 3b shows: A bone joining device 400 and a hollow fixation part are formed as a unibody, a body 130 having an annular texture, an injecting end 120 having a plurality of holes for injecting a medical filling into a cover part via the injecting end 120. FIG. 3c shows: A bone joining device 400 and a hollow fixation part are formed as a unibody, a body 130 having a striped texture, an injecting end 120 having a plurality of holes for injecting a medical filling into a cover part via the injecting end 120. FIG. 3d shows: A bone joining device 400 and a hollow fixation part are formed as a unibody, a body 130 having a speckled texture, an injecting end 120 having a plurality of holes for injecting a medical filling into a cover part via the injecting end 120. FIG. 3e shows: A bone joining device 400 and a hollow fixation part are formed as a unibody, a body 130 having a mesh-like texture, an injecting end 120 having a plurality of holes for injecting a medical filling into a cover part via the injecting end 120.

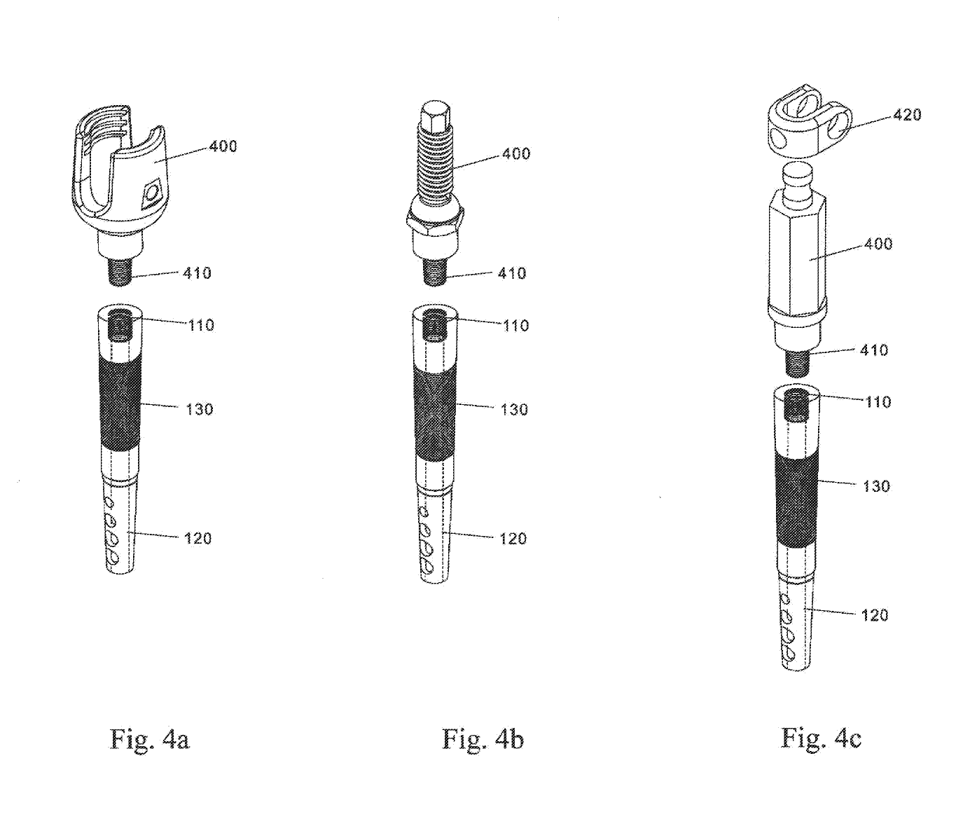

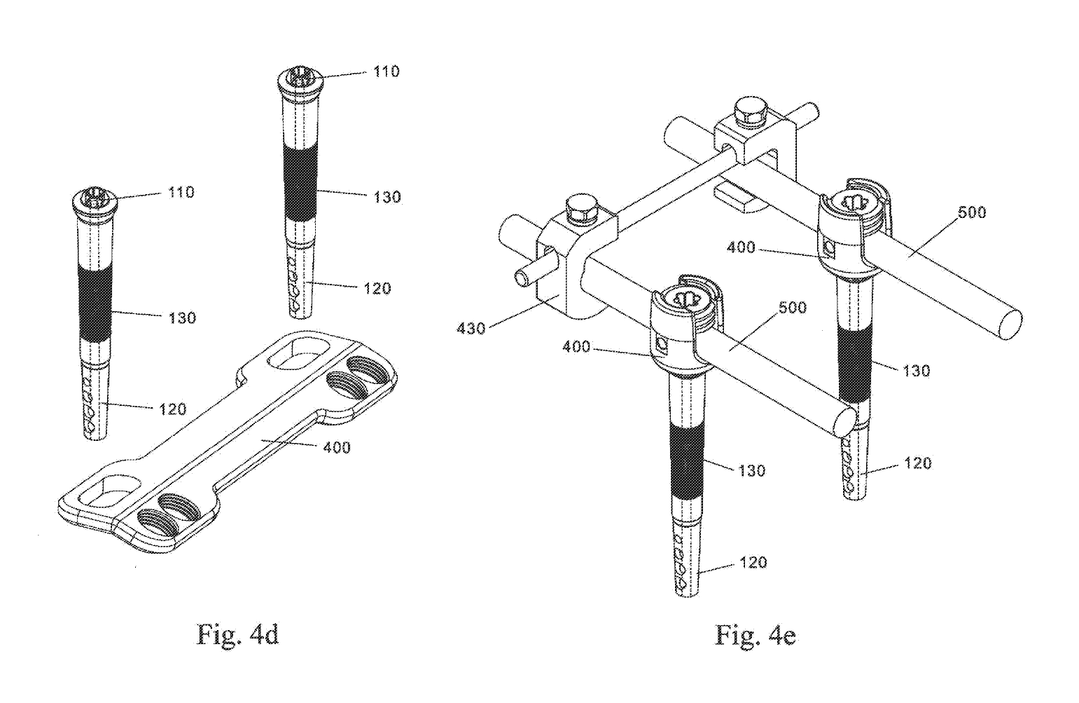

FIGS. 4a, 4b, 4c, 4d, and 4e are schematic views showing the joining between the bone fixation device with four types of bone joining device to form a spine fixation system, according to a preferred embodiment of the present invention. FIG. 4a shows: A bone joining device 400 being a receiving trough and may be screwed together with a hollow fixation part, a body 130 having a mesh-like texture, an injecting end 120 having a plurality of holes for injecting a medical filling into a cover part via the injecting end 120. FIG. 4b shows: A bone joining device 400 being a joining rod and may be screwed together with a hollow fixation part, a body 130 having a mesh-like texture, an injecting end 120 having a plurality of holes for injecting a medical filling into a cover part via the injecting end 120. FIG. 4c shows: A bone joining device 400 being a joining rod, the joining rod 400 is joined with a tightening device 420 and may be screwed together with a hollow fixation part, a body 130 having a mesh-like texture, an injecting end 120 having a plurality of holes for injecting a medical filling into a cover part via the injecting end 120. FIG. 4d shows: A bone joining device 400 being a bone plate and may be screwed together with a hollow fixation part, a body 130 having a mesh-like texture, an injecting end 120 having a plurality of holes for injecting a medical filling into a cover part via the injecting end 120. FIG. 4e shows: A bone joining device 400 being a receiving trough, the receiving trough 400 is joined with holders 430 by using rods 500, and may be screwed together with hollow fixation parts, a body 130 having a mesh-like texture, an injecting end 120 having a plurality of holes for injecting a medical filling into a cover part via the injecting end 120.

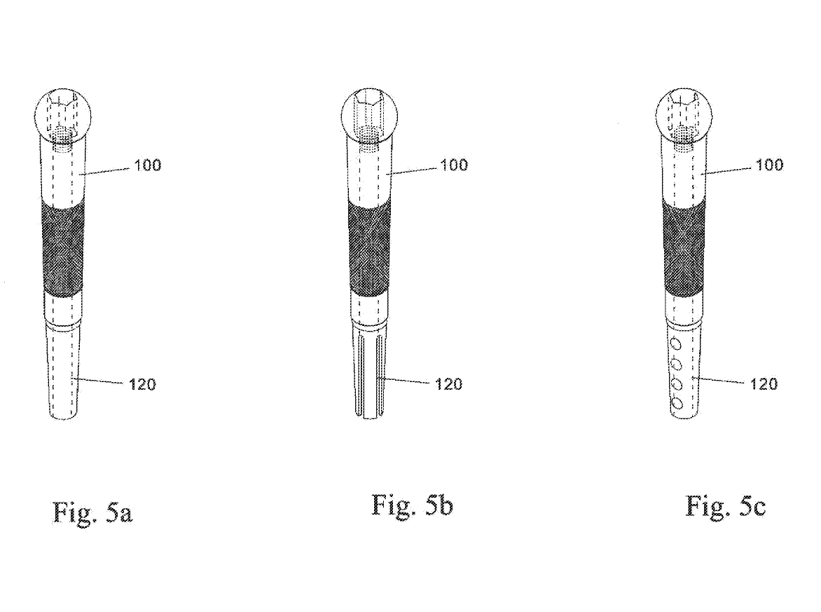

FIGS. 5a, 5b, and 5c are schematic views showing three types of injecting end of the hollow fixation device of the bone fixation device according to a preferred embodiment of the present invention. Wherein 100 is a hollow fixation part, and 120 is an injecting end. FIG. 5a shows: The injecting end 120 having no holes or slits. FIG. 5b shows: The injecting end 120 having a plurality of slits for injecting a medical filling into a cover part via the injecting end 120. FIG. 5c shows: The injecting end 120 having a plurality of holes for injecting a medical filling into a cover part via the injecting end 120.

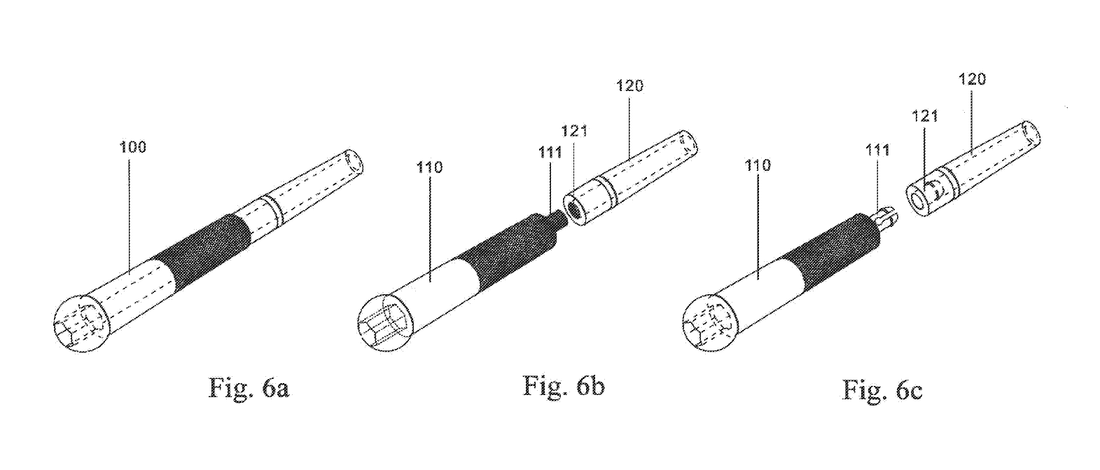

FIGS. 6a, 6b, and 6c are schematic views showing three types of coupling for the hollow fixation device of the bone fixation device according to a preferred embodiment of the present invention. Wherein 100 is a hollow fixation part, 110 is a joining end, 111 is a coupling end of the joining end 110 120 is an injecting end, and 121 is a coupling end of the injecting end 120. FIG. 6a shows: The hollow fixation part 100 being formed as a unibody. FIG. 6b shows: The joining end 110 being coupled to the injecting end 120 by screwing. FIG. 6c shows: The joining end 110 being coupled to the injecting end 120 by clasping.

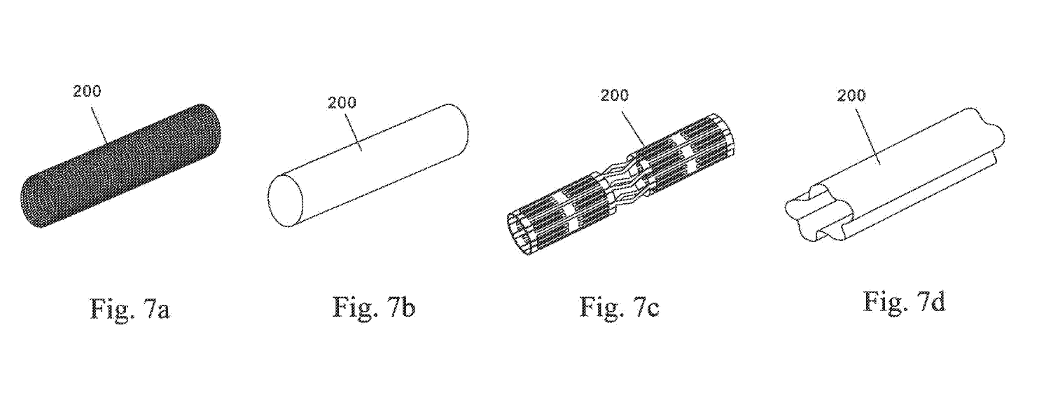

FIGS. 7a, 7b, 7c, and 7d are schematic views showing four types of cover part for the bone fixation device according to a preferred embodiment of the present invention. Wherein 200 is a cover part. FIG. 7a shows: The cover part 200 being mesh-like. FIG. 7b shows: The cover part 200 being made of PET materials. FIG. 7c shows: The cover part 200 being made of metal. FIG. 7d shows: The cover part 200 being elastic.

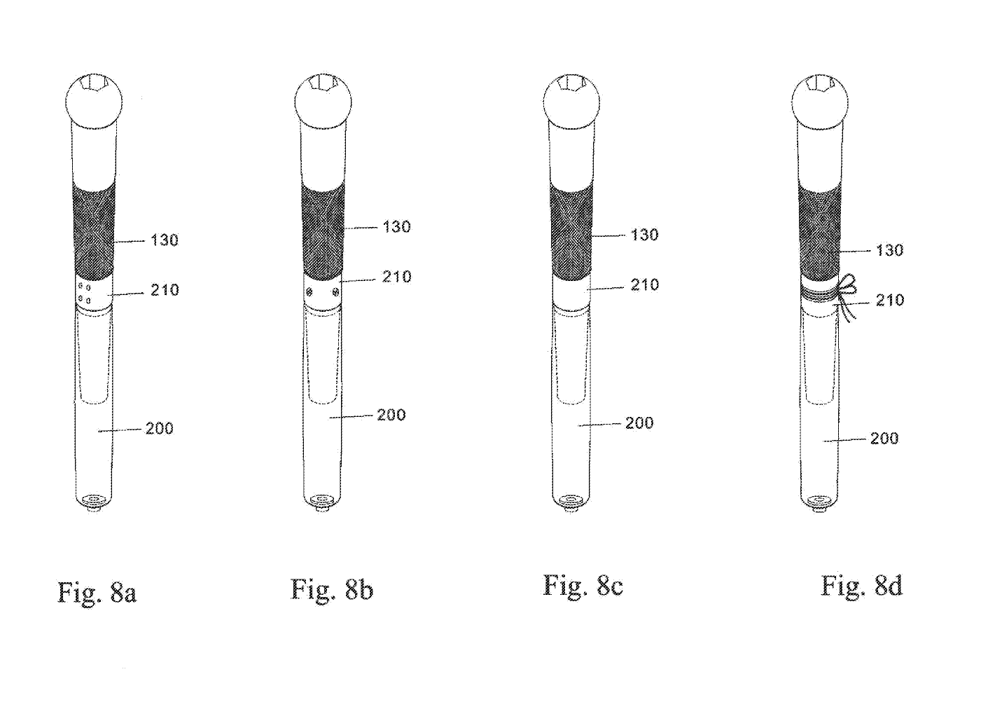

FIGS. 8a, 8b, 8c, and 8d are schematic views showing the joining between four types of cover part with the hollow fixation part of the bone fixation device according to a preferred embodiment of the present invention. Wherein 130 is a body, 200 is a cover part, and 210 is a securing ring. FIG. 8a shows: The body 130 being coupled to the cover part 200 by riveting. FIG. 8b shows: The body 130 being coupled to the cover part 200 by screwing. FIG. 8c shows: The body 130 being coupled to the cover part 200 by tightening. FIG. 8d shows: The body 130 being coupled to the cover part 200 by binding.

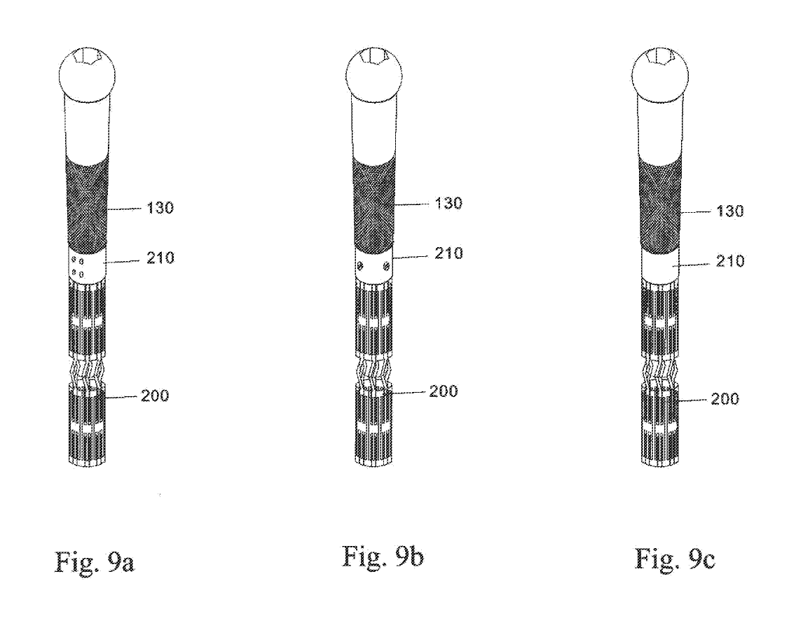

FIGS. 9a, 9b, and 9c are schematic views showing the joining between three types of metal cover part with the hollow fixation device of the bone fixation device according to a preferred embodiment of the present invention. Wherein 130 is a body, 200 is a cover part, and 210 is a securing ring. FIG. 9a shows: The cover part 200 being made of metal, and the body 130 being coupled to the cover part 200 by riveting. FIG. 9b shows: The cover part 200 being made of metal, and the body 130 being coupled to the cover part 200 by screwing. FIG. 9c shows: The cover part 200 being made of metal, and the body 130 being coupled to the cover part 200 by tightening.

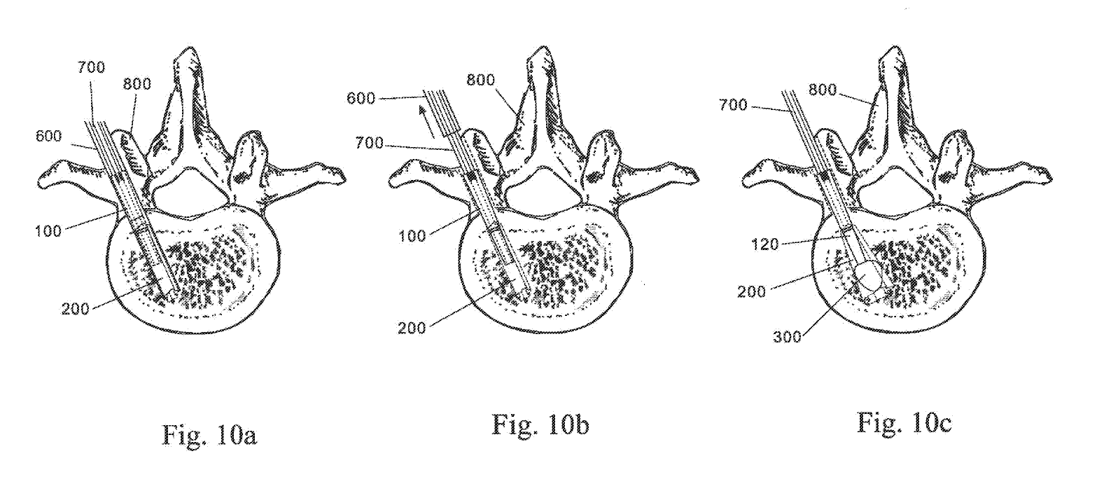

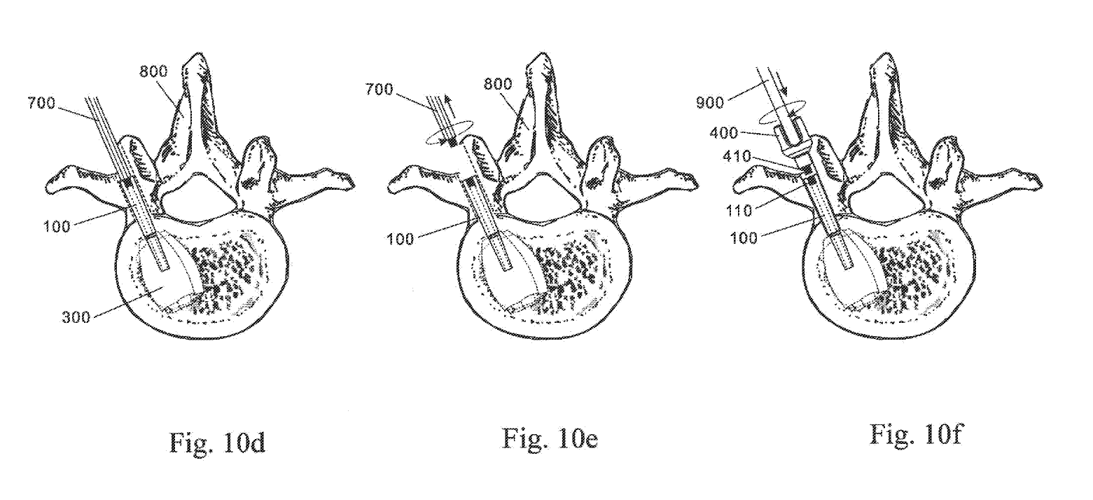

FIGS. 10a, 10b, 10c, 10d, 10e, and 10f are schematic views showing the surgical steps for joining the bone fixation device of the invention with a bone joining device. Wherein 100 is a hollow fixation part, 120 is an injecting end, 200 is a cover part, 300 is a medical filling, 400 is a bone joining device, 410 is a coupling end, 600 is a tube, 700 is an injecting device, 800 is a vertebra, and 900 is a combined device. FIG. 10a shows: The injecting device 700, the hollow fixation part 100, and the cover part 200 are sequentially placed into the tube 600, and then the tube 600 is implanted into the vertebra 800 where a hole has been created. FIG. 10b shows: The injecting device 700, the hollow fixation part 100, and the cover part 200 are implanted into the vertebra 800 where a hole has been created, then the tube 600 is removed. FIG. 10c shows: The medical filling 300 is subsequently injected into the hollow fixation part 100 via the injecting device 700, and then into the cover part 200 via the injecting end 120. FIG. 10d shows: The medical filling 300 and the cover part 200 have become lump-like. FIG. 10e shows: After injecting is completed, the injecting device 700 is removed. FIG. 10f shows: The bone joining device 400 is then locked into the hollow fixation part 100.

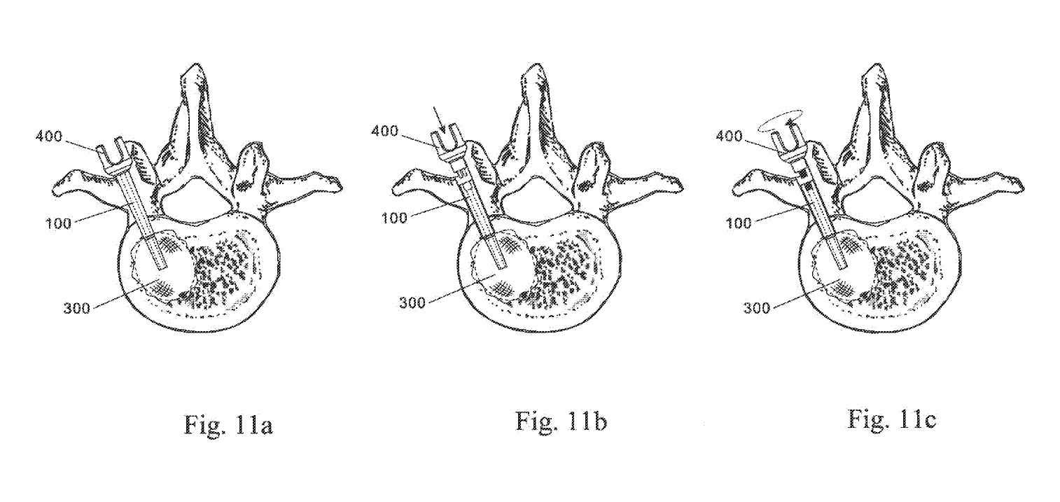

FIGS. 11a, 11b, and 11c are schematic views showing the joining between the bone fixation device of the invention with a bone joining device having a tightening mechanism. Wherein 100 is a hollow fixation part, 300 is a medical filling, and 400 is a bone joining device. FIG. 11a shows: The lump-like medical filling 300 fixes a spine fixation system on the vertebra along with a unibody of the hollow fixation part 100 and the bone joining device 400. FIG. 11b shows: The spine fixation system fixes itself on the vertebra by using a clasping-type tightening mechanism between the hollow fixation part 100 and the bone joining device 400. FIG. 11c shows: The spine fixation system fixes itself on the vertebra by using a screwing-type tightening mechanism between the hollow fixation part 100 and the bone joining device 400.

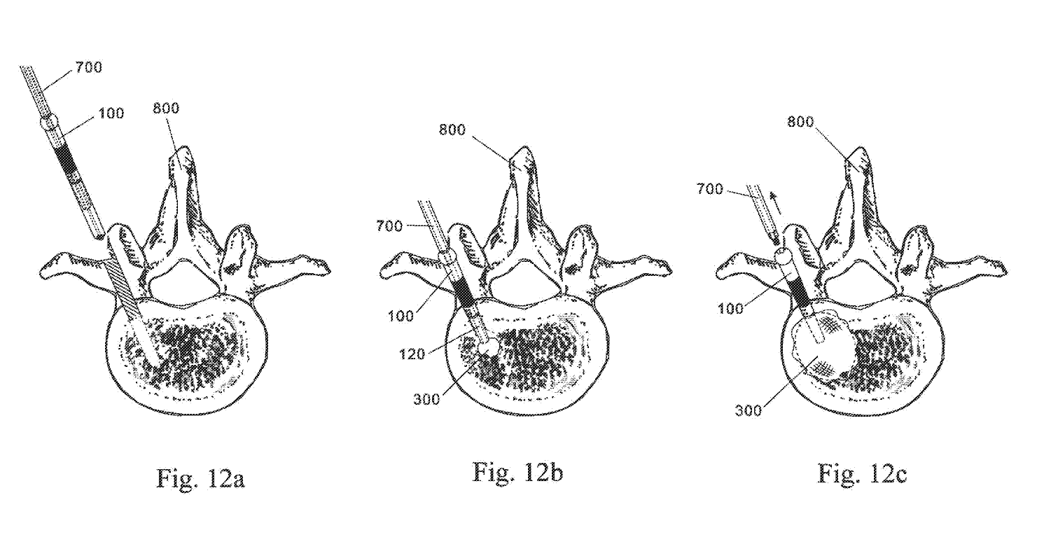

FIGS. 12a, 12b, and 12c are schematic views showing the surgical steps for joining the bone fixation device of the invention with a cover part made of PET. Wherein 100 is a hollow fixation part, 120 is an injecting end, 300 is a medical filling, 700 is an injecting device, and 800 is a vertebra. FIG. 12a shows: The hollow fixation part 100 is implanted into the vertebra 800 where a hole has been created by employing the injecting device 700. FIG. 12b shows: The medical filling 300 is subsequently injected into the hollow fixation part 100 via the injecting device 700, and then into the cover part via the injecting end 120. FIG. 12c shows: The medical filling 300 has become lump-like completely.

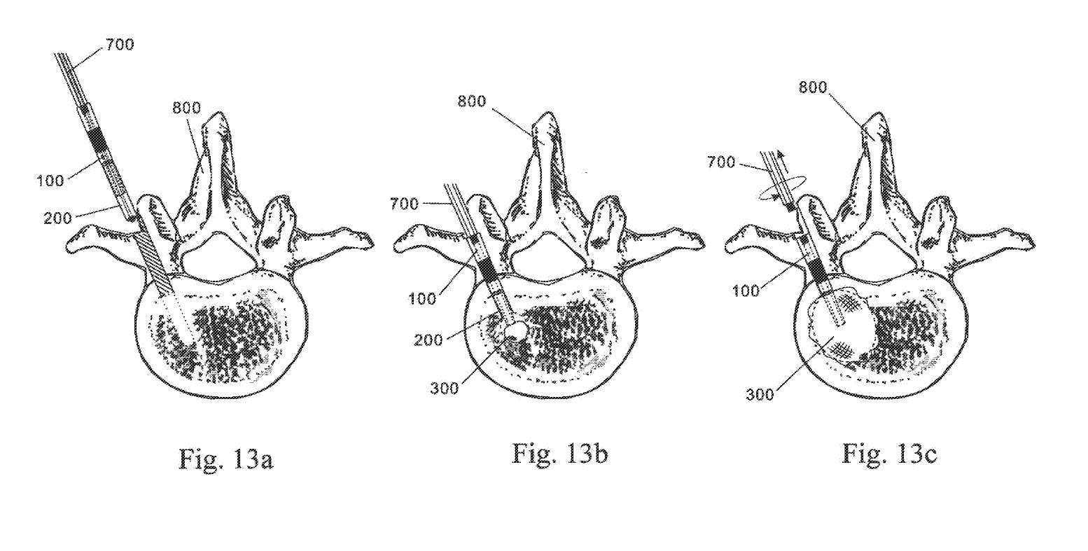

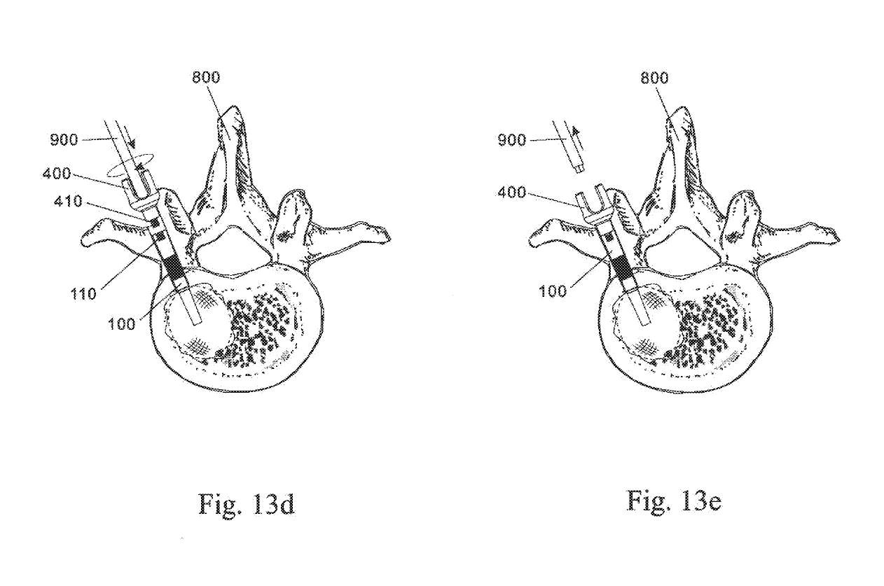

FIGS. 13a, 13b, 13c, 13d, and 13e are schematic views showing the surgical steps for joining the bone fixation device of the invention with a bone joining device having a tightening mechanism. Wherein 100 is a hollow fixation part, 200 is a cover part, 300 is a medical filling, 400 is a bone joining device, 700 is an injecting device, 800 is a vertebra, and 900 is a combined device. FIG. 13a shows: The hollow fixation part 100 is implanted into the vertebra 800 where a hole has been created by utilizing the injecting device 700. FIG. 13b shows: The medical filling 300 is then injected into the hollow fixation part 100 via the injecting device 700, and then into the cover part via the injecting end. FIG. 13c shows: The medical filling 300 has become lump-like completely. FIG. 13d shows: The bone joining device 400 is then locked into the hollow fixation part 100. FIG. 13e shows: The bone joining device 400 has been fully combined into the hollow fixation part 100.

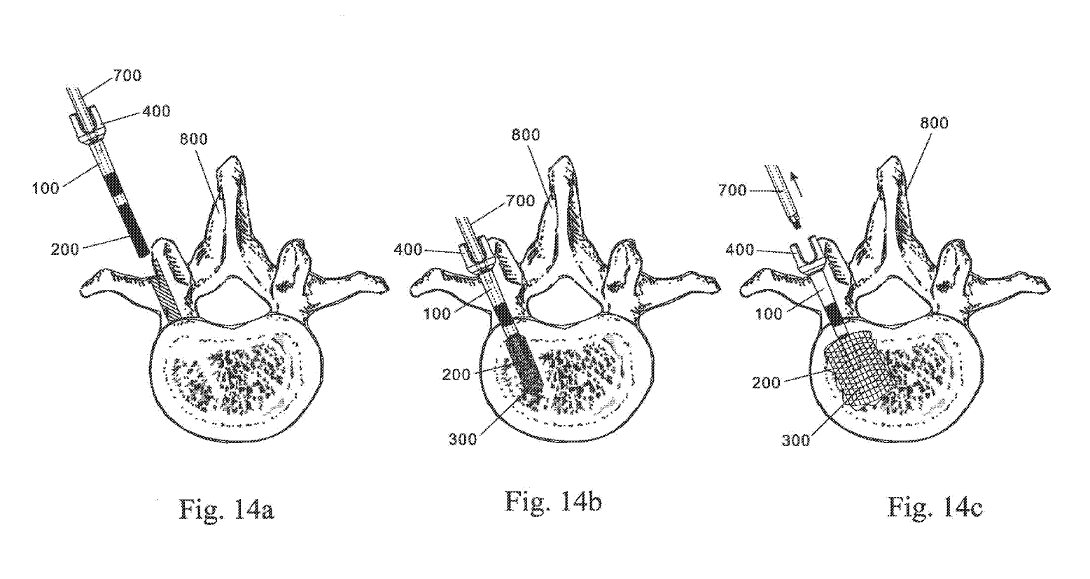

FIGS. 14a, 14b, and 14c are schematic views showing the surgical steps for joining the bone fixation device of the invention with an elastic metal cover part. Wherein 100 is a hollow fixation part, 200 is a cover part, 300 is a medical filling, 400 is a bone joining device, 700 is an injecting device, and 800 is a vertebra. FIG. 14a shows: The cover part 200 is made of metal, and the injecting device 700 is used to implant the hollow fixation part 100 into the vertebra 800 where a hole has been created. FIG. 14b shows: The medical filling 300 is then injected into the hollow fixation part 100 via the injecting device 700, and then into the metal cover part 200 via the injecting end, so as to push open the metal cover part. FIG. 14c shows: The medical filling 300 has been completely injected and pushed open the metal cover part 200 so it becomes lump-like.

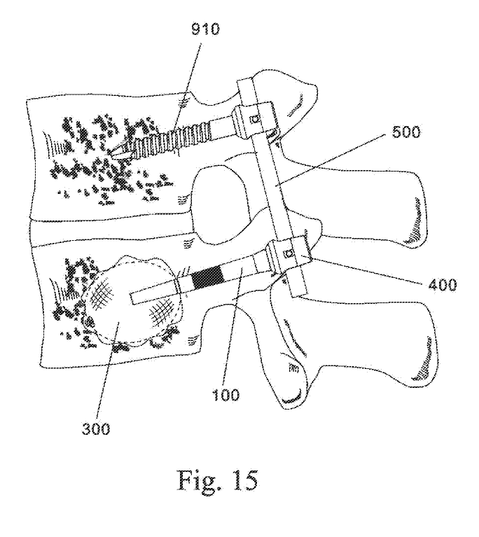

FIG. 15 is a schematic view showing a spine fixation system comprising the bone fixation device of the invention being joined with a bone joining device. Wherein 100 is a hollow fixation part, 300 is a medical filling, 400 is a bone joining device, 500 is a rod, and 910 is a bone nail. The hollow fixation part 100 is joined to the bone nail 910 by using the bone joining device 400 and the rod 500.

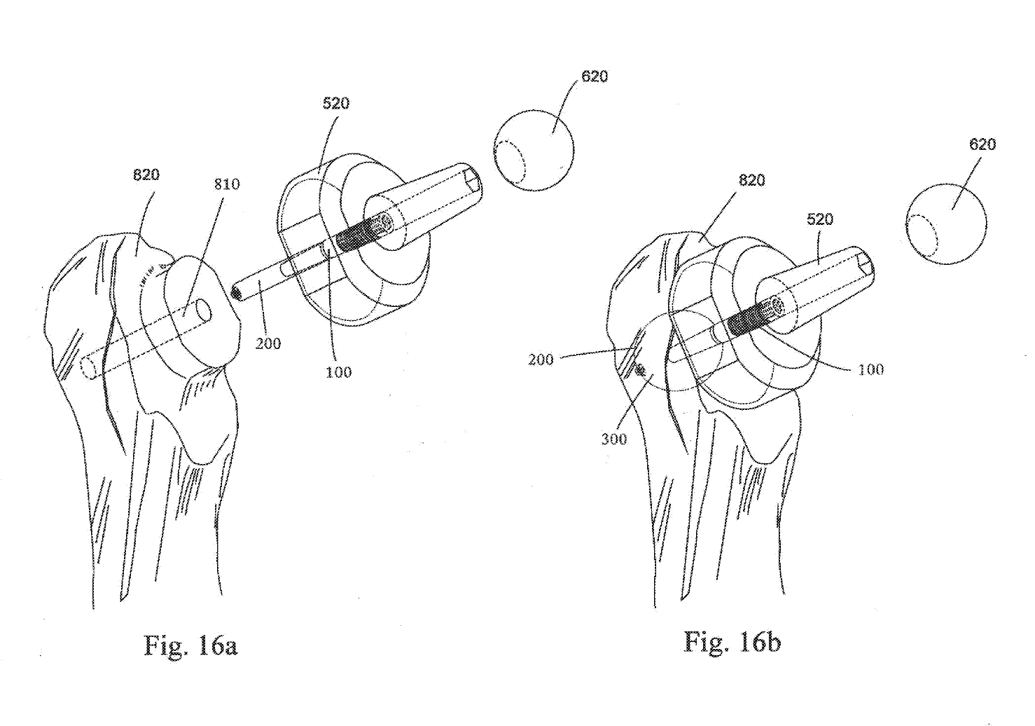

FIG. 16a is a schematic view showing an artificial hip joint replacement system comprising the bone fixation device being joined with a hip joint substitute and an acetabular implant, according to a preferred embodiment of the present invention. Wherein 100 is a hollow fixation part, 200 is a cover part, 520 is a hip joint substitute, 620 is an acetabular implant, 820 is a hip joint, and 810 is a hole. The hollow fixation part 100 and the hip joint substitute 520 are formed as a unibody. During surgery, the hollow fixation part 100 and the cover part 200 are placed into the hole 810 of the hip joint 820 created beforehand.

FIG. 16b is a schematic view showing the system of FIG. 16a after injecting is completed according to a preferred embodiment of the present invention. Wherein 100 is a hollow fixation part, 200 is a cover part, 300 is a medical filling, 520 is a hip joint substitute, 620 is an acetabular implant, and 820 is a hip joint. Moreover, the medical filling 300 is injected into the cover part 200 via the injecting end of the hollow fixation part 100, and the medical filling 300 is allowed to completely fill up the cover part 200 and become lump-like.

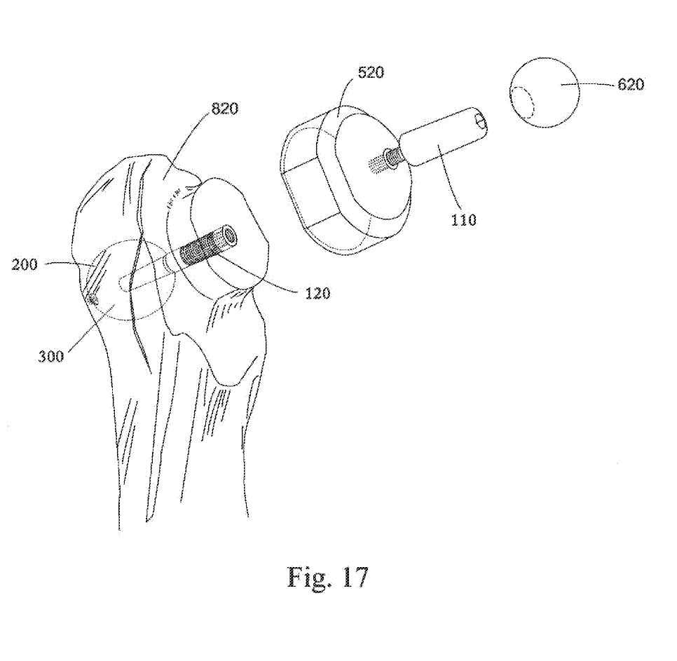

FIG. 17 is a schematic view showing an artificial hip joint replacement system comprising the bone fixation device being joined with a hip joint substitute and an acetabular implant, according to another preferred embodiment of the present invention. Wherein 110 is a joining end, 120 is an injecting end, 200 is a cover part, 520 is a hip joint substitute, 620 is an acetabular implant, and 820 is a hip joint. A hollow fixation part and the hip joint substitute 520 are formed as two parts and joined together by screwing. During surgery, the hollow fixation part 100 and the cover part 200 are placed into the hole 810 of the hip joint 820 created beforehand.

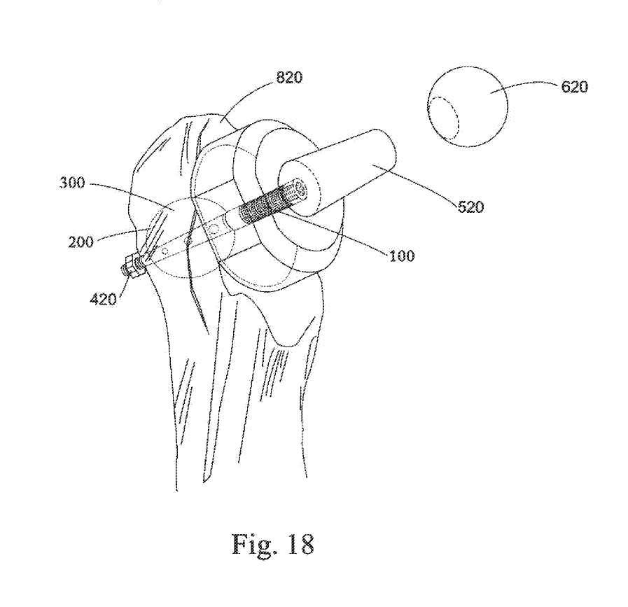

FIG. 18 is a schematic view showing an artificial hip joint replacement system comprising the bone fixation device being joined with a hip joint substitute and an acetabular implant, according to yet another preferred embodiment of the present invention. Wherein 100 is a hollow fixation part, 200 is a cover part, 300 is a medical filling, 520 is a hip joint substitute, 620 is an acetabular implant, 420 is a tightening device, and 820 is a hip joint. After injecting the medical filling into this artificial hip joint replacement system, a tightening device 420 is used to push and tighten the lump-like cover part toward the bone of surgical site.

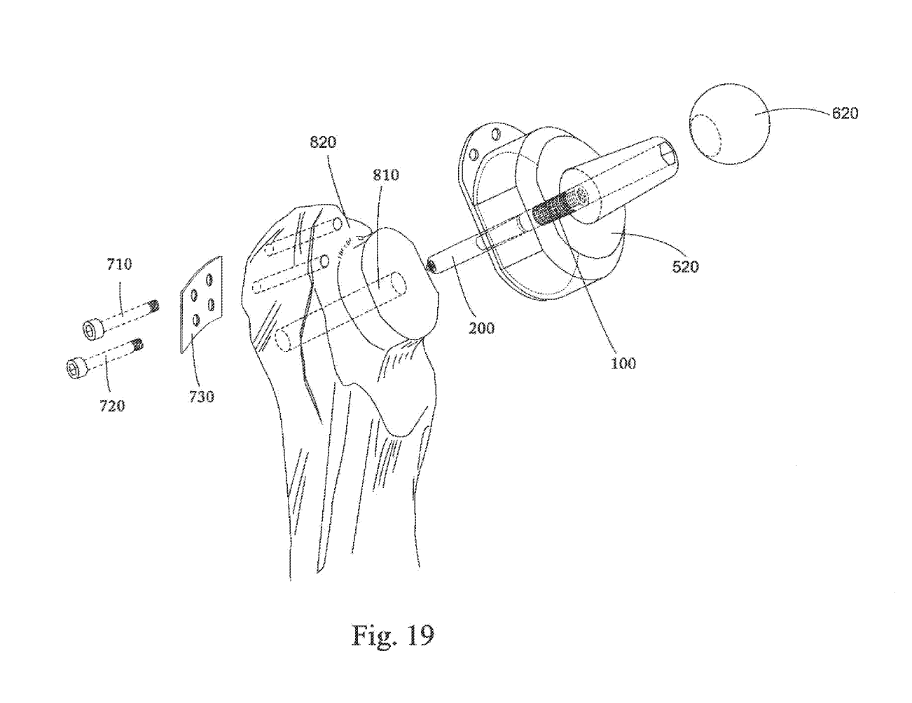

FIG. 19 is a schematic view showing an artificial hip joint replacement system comprising the bone fixation device being joined with a hip joint substitute and an acetabular implant, according to still another preferred embodiment of the present invention. Wherein 100 is a hollow fixation part, 200 is a cover part, 520 is a hip joint substitute, 620 is an acetabular implant, 710 is a first screw, 720 is a second screw, 730 is a plate. 820 is a hip joint, and 810 is a hole. After injecting the medical filling into this artificial hip joint replacement system, the first screw 710, the second screw 720, and the plate 730 are used to push and tighten the lump-like cover part toward the bone of surgical site.

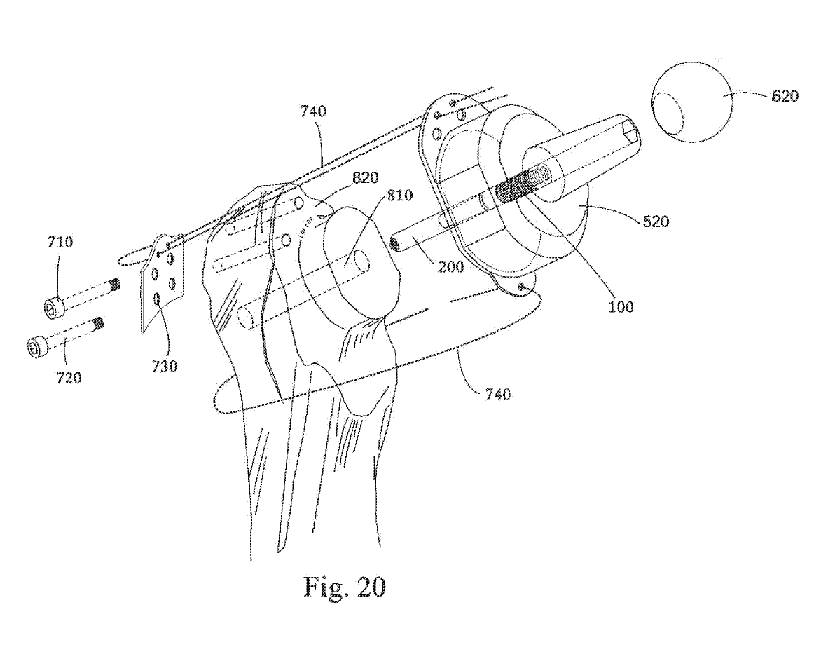

FIG. 20 is a schematic view showing an artificial hip joint replacement system of the invention being joined with screws and a securing cable, according to a preferred embodiment of the present invention. Wherein 100 is a hollow fixation part, 200 is a cover part, 520 is a hip joint substitute, 620 is an acetabular implant, 710 is a first screw, 720 is a second screw, 730 is a plate, 740 is a securing cable, 820 is a hip joint, and 810 is a hole. After injecting the medical filling into the artificial hip joint replacement system, the first screw 710, the second screw 720, the plate 730, and the securing cable 740 are used to push and tighten the lump-like cover part toward the bone of surgical site.

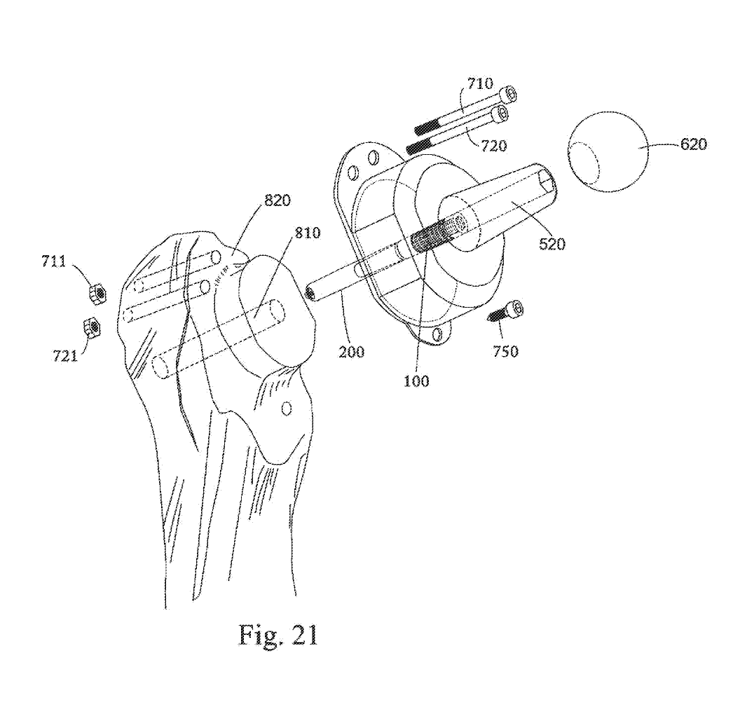

FIG. 21 is a schematic view showing an artificial hip joint replacement system of the invention being joined with screws, according to another preferred embodiment of the present invention. Wherein 100 is a hollow fixation part, 200 is a cover part, 520 is a hip joint substitute, 620 is an acetabular implant. 710 is a first screw, 711 is a first nut, 720 is a second screw, 721 is a second nut, 750 is a third screw, 820 is a hip joint, and 810 is a hole. After injecting the medical filling into the artificial hip joint replacement system, the first screw 710, the second screw 720, the first nut 711, the second nut 721, and the third screw 750 are used to push and tighten the lump-like cover part toward the bone of surgical site.

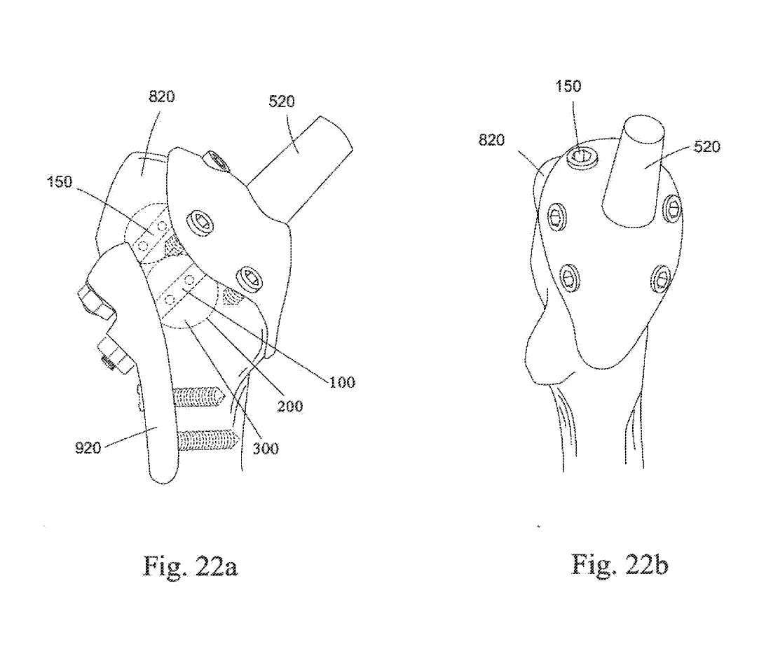

FIG. 22a is a schematic view showing an artificial hip joint replacement system of the invention being joined with a second hollow fixation part, according to a preferred embodiment of the present invention. Wherein 100 is a hollow fixation part, 200 is a cover part, 300 is a medical filling, 150 is a second hollow fixation part, 520 is a hip joint substitute, 820 is a hip joint, and 920 is a securing plate. The artificial hip joint replacement system is internally reinforced by using the second hollow fixation part 150 and the cover part to provide additional space for injecting the medical filling 300, and also externally reinforced by further using the securing plate 920 and screws.

FIG. 22b is a schematic view showing the system of FIG. 22a from another angle of view.

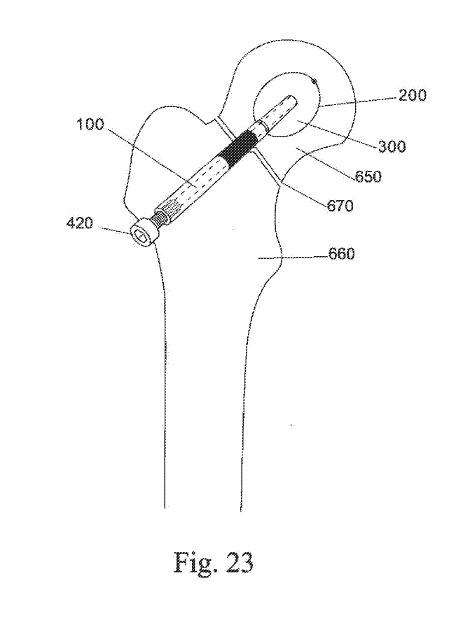

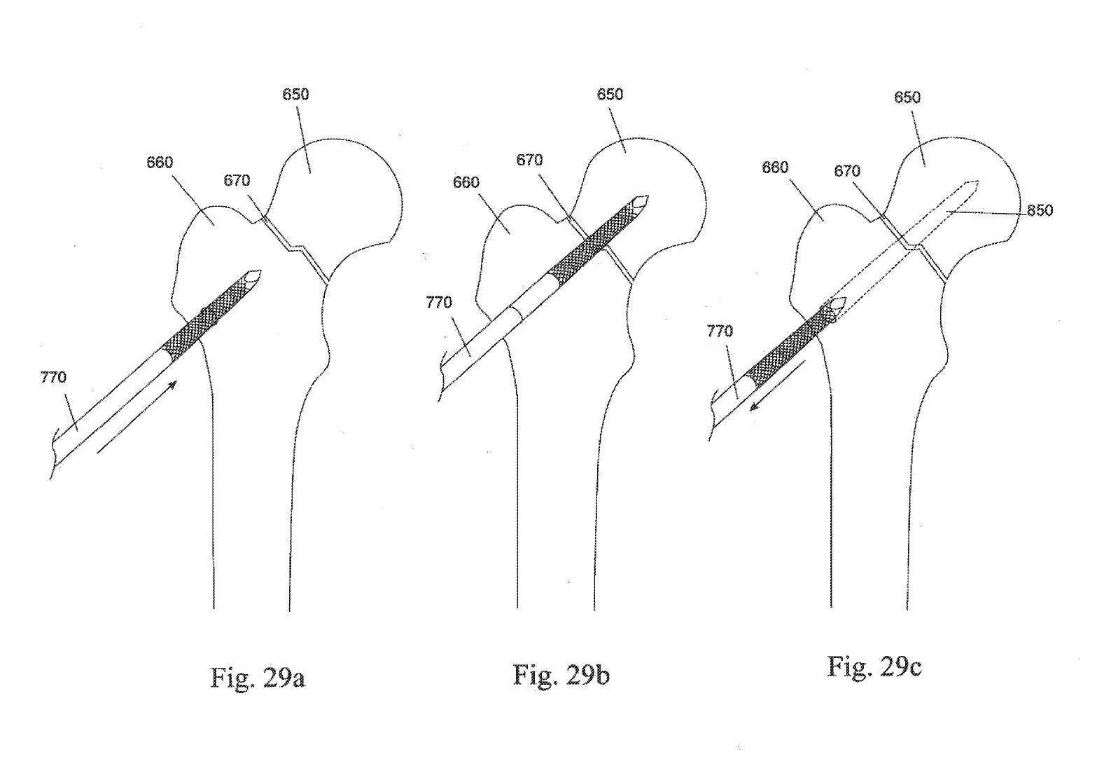

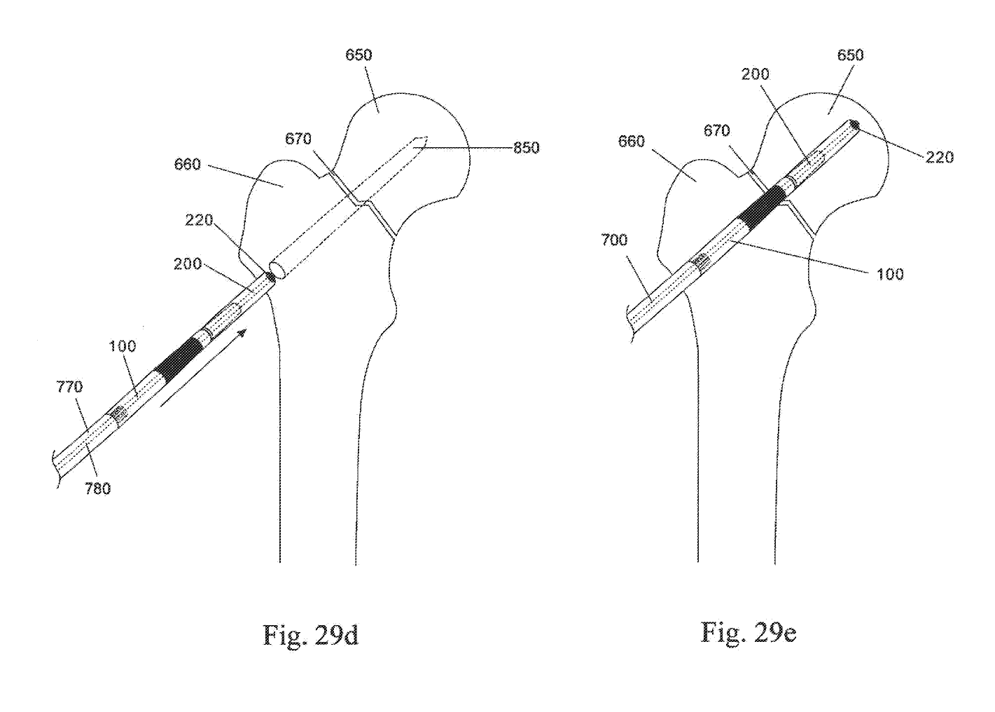

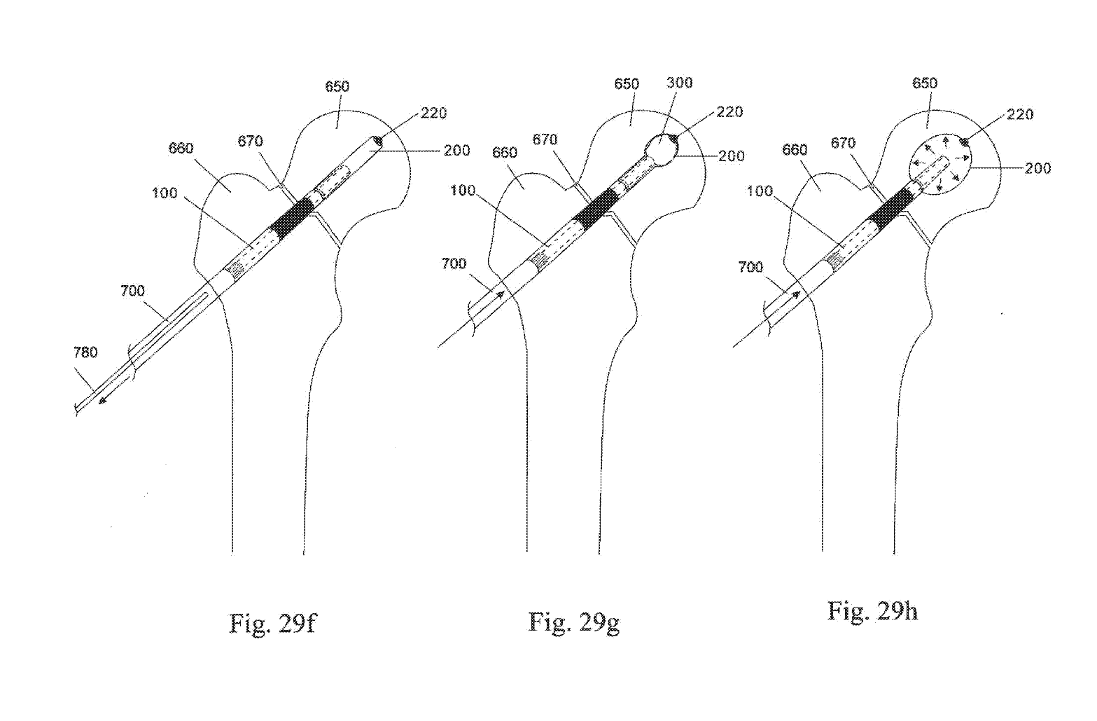

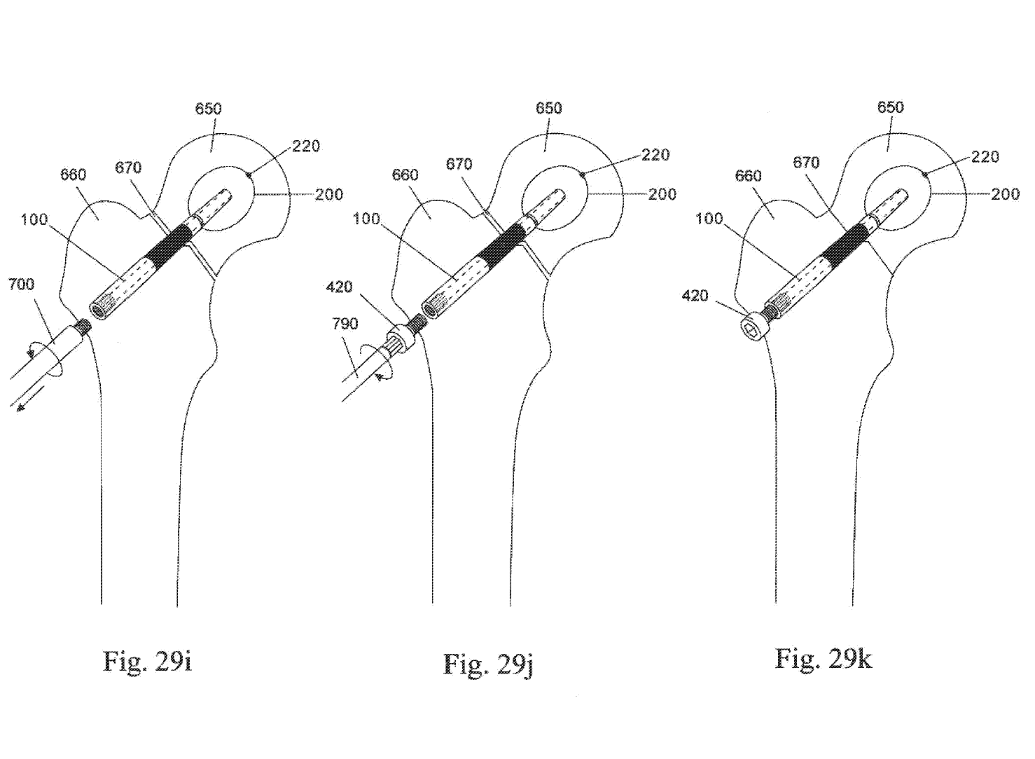

FIG. 23 is a schematic view showing a long bone fixation system comprising the bone fixation device being joined with a tightening device, according to a preferred embodiment of the present invention. Wherein 100 is a hollow fixation part, 200 is a cover part, 300 is a medical filling, 420 is a tightening device, 650 is a front broken bone, 660 is a rear broken bone, and 670 is a split. The hollow fixation part 100 and the cover part 200 are fitted through the rear broken bone 660, the split 670, and the front broken bone 650, and the front and rear broken bones 650 and 660 are rejoined by injecting the medical filling 300. The tightening device 420 is further employed to reinforce the joining between the front broken bone 650, the rear broken bone 660, and the hollow fixation part 100 in order to enhance the stability thereof. The surgical method thereof is further described in FIGS. 29a to 29k.

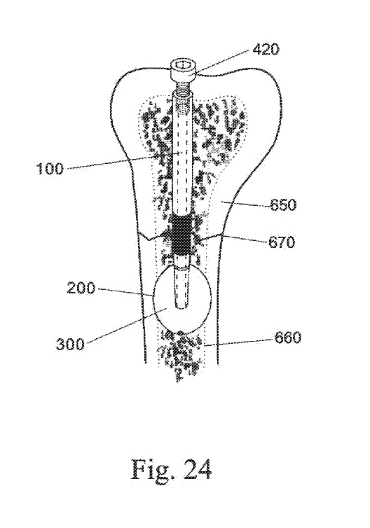

FIG. 24 is a schematic view showing a long bone fixation system comprising the bone fixation device being joined with a tightening device, according to another preferred embodiment of the present invention. Wherein 100 is a hollow fixation part, 200 is a cover part, 300 is a medical filling, 420 is a tightening device, 650 is a front broken bone, 660 is a rear broken bone, and 670 is a split. The hollow fixation part 100 and the cover part 200 are fitted through the front broken bone 650, the split 670, and the rear broken bone 660, and the front and rear broken bones 650 and 660 are rejoined by injecting the medical filling 300. The tightening device 420 is further employed to reinforce the joining between the front broken bone 650, the rear broken bone 660, and the hollow fixation part 100 in order to enhance the stability thereof. The surgical method thereof is further described in FIGS. 29a to 29k.

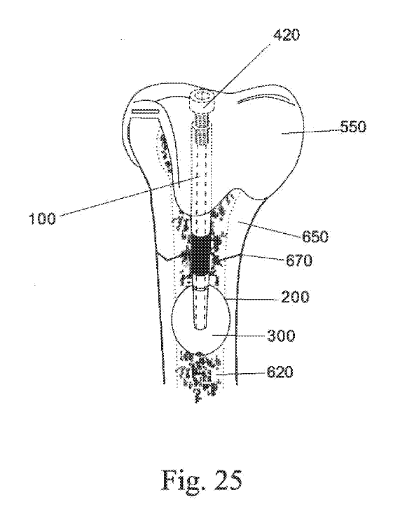

FIG. 25 is a schematic view showing a long bone fixation system comprising the bone fixation device being joined with a tightening device, according to yet another preferred embodiment of the present invention. Wherein 100 is a hollow fixation part, 200 is a cover part, 300 is a medical filling, 420 is a tightening device, 550 is a supplementary fixation device, 650 is a front broken bone, 660 is a rear broken bone, and 670 is a split. The hollow fixation part 100 and the cover part 200 are fitted through the supplementary fixation device 550, the front broken bone 650, the split 670, and the rear broken bone 660, and the front and rear broken bones 650 and 660 are rejoined by injecting the medical filling 300. The tightening device 420 is further employed to reinforce the joining between the front broken bone 650, the rear broken bone 660, and the hollow fixation part 100 in order to enhance the stability thereof. The surgical method thereof is further described in FIGS. 29a to 29k.

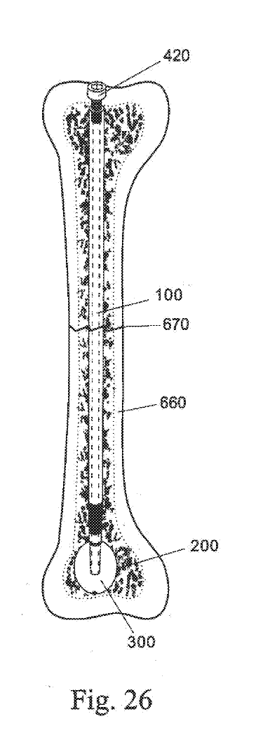

FIG. 26 is a schematic view showing a long bone fixation system comprising the bone fixation device being joined with a tightening device, according to yet another preferred embodiment of the present invention. Wherein 100 is a hollow fixation part, 200 is a cover part, 300 is a medical filling, 420 is a tightening device, 650 is a front broken bone, 660 is a rear broken bone, and 670 is a split. The hollow fixation part 100 and the cover part 200 are fitted through the front broken bone 650, the split 670, and the rear broken bone 660, and the front and rear broken bones 650 and 660 are rejoined by injecting the medical filling 300. The tightening device 420 is further employed to reinforce the joining between the front broken bone 650, the rear broken bone 660, and the hollow fixation part 100 in order to enhance the stability thereof. The surgical method thereof is further described in FIGS. 29a to 29k.

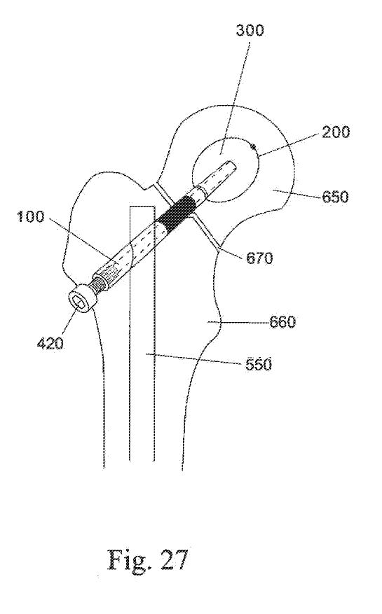

FIG. 27 is a schematic view showing a long bone fixation system comprising the bone fixation device being joined with a tightening device, according to still another preferred embodiment of the present invention. Wherein 100 is a hollow fixation part, 200 is a cover part. 300 is a medical filling. 420 is a tightening device, 550 is a supplementary fixation device, 650 is a front broken bone, 660 is a rear broken bone, and 670 is a split. The hollow fixation part 100 and the cover part 200 are fitted through the rear broken bone 660, the supplementary fixation device 550, the split 670, and the front broken bone 650, and the front and rear broken bones 650 and 660 are rejoined by injecting the medical filling 300. The tightening device 420 is further utilized to reinforce the joining between the front broken bone 650, the rear broken bone 660, and the hollow fixation part 100 in order to enhance the stability thereof. The surgical method thereof is further described in FIGS. 29a to 29k.

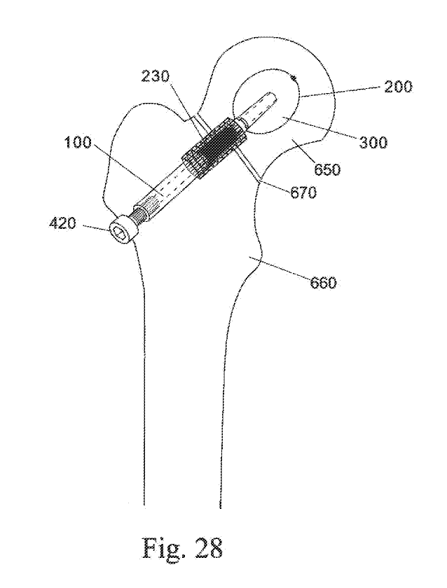

FIG. 28 is a schematic view showing a long bone fixation system comprising the bone fixation device being joined with a tightening device, according to a further preferred embodiment of the present invention. Wherein 100 is a hollow fixation part, 200 is a cover part, 230 is a second cover part, 300 is a medical filling, 420 is a tightening device, 650 is a front broken bone, 660 is a rear broken bone, and 670 is a split. The hollow fixation part 100. the cover part 200, and the second cover part 230 are fitted through the rear broken bone 660, the split 670, and the front broken bone 650, then the front and rear broken bones 650 and 660 are secured together by injecting the medical filling 300 into the cover part 200, and then reinforced by injecting the medical filling 300 into the second cover part 230. The tightening device 420 is further utilized to reinforce the joining between the front broken bone 650, the rear broken bone 660, and the hollow fixation part 100 in order to enhance the stability thereof.

FIGS. 29a to 29k are schematic views showing the surgical steps for installing the long bone fixation system of the invention. Wherein 100 is a hollow fixation part, 200 is a cover part, 220 is a halting part, 300 is a medical filling, 420 is a tightening device, 650 is a front broken bone, 660 is a rear broken hone, 670 is a split, 770 is a drilling device, 780 is a guiding device, 700 is an injecting device, and 790 is a locking device. FIG. 29a shows: The drilling device 770 drilling into the rear broken bone 660. FIG. 29b shows: The drilling device 770 drilling into the rear broken bone 660, via the split 670, and into the front broken bone 650. FIG. 29c shows: The drilling device 770 being removed after drilling is completed. FIG. 29d shows: The hollow fixation part 100 and the cover part 200 are placed into a hole 850 created by the drilling device 770, wherein the injecting device 700 is joined with the hollow fixation part 100, and the guiding device 780 is allowed to go through the hollow fixation part 100 via the injecting device 700, into the cover part 200 and push against the halting part 220, so as to support and protect the cover part 200 from collapsing when it is inserted into the broken bone. FIG. 29e shows: The hollow fixation part 100 and the cover part 200 being completely inserted into the hole 850. FIG. 29f shows: The long bone fixation system being placed into position, and the guiding device 780 is then removed from the system. FIG. 29g shows: The medical filling 300 is injected into the cover part 200 via the injecting device 700 and the hollow fixation part 100. FIG. 29h shows: The medical filling 300 and the cover part 200 have become lump-like. FIG. 29i shows: The injecting device 700 being removed from the hollow fixation part 100 after injecting is completed. FIG. 29j shows: The tightening device 420 being locked into the hollow fixation part 100 by using the locking device 790. FIG. 29k shows: The tightening device 420 being locked into the hollow fixation part 100 and used to tighten the joining between the front broken bone 650, the rear broken bone 660, and the hollow fixation part.

What is claimed is:

1. A method for operating on a spine by using a spinal fixation device comprising a hollow fixation part having a body, a joining end at a proximal end of the body, and an injecting end at distal end of the body; a cover part being joined with the injecting end of said hollow fixation part; a medical filling being forceable and/or injectable into said cover part via the injecting end of said hollow fixation part such that the medical filling is allowed to become lump-like after injecting is completed; wherein said cover part is joined with the injecting end of said hollow fixation part such that said cover part, said injecting end and said joining end align in a same longitudinal direction;
wherein said cover part is adapted to be implanted inside a vertebra with said joining end outside the vertebra; said method comprising the steps of:
(1) creating a hole on a vertebra;
(2) placing said hollow fixation part joined with said cover part into the hole, and leaving the joining end of said hollow fixation part outside of the vertebra and exposed;
(3) injecting said medical filling into said cover part via an injecting device and said hollow fixation part;
(4) allowing said medical filling to become solidified so that said medical filling and the cover part form a lump;
(5) removing said injecting device from said hollow fixation part.

2. The method of claim 1 further comprising a step (6) of joining a bone joining device with the joining end of said hollow fixation part.

3. The method of claim 1 further comprising repeating step (1) to (5) to another vertebra with another one of said spinal fixation device, and joining the joining ends of the hollow fixation parts of the two spinal fixation devices with a bone joining device.

4. The method of claim 3, wherein said bone joining device is a rod, a bone plate, or a joining rod.

5. The method of claim 1, wherein the body of said hollow fixation part is of annular textures, striped textures, speckled textures, or mesh-like textures.

6. The method of claim 1, wherein the injecting end of said hollow fixation part has a plurality of slits or holes disposed thereon.

7. The method of claim 1, wherein the body of said hollow fixation part has an annular trough disposed thereon.

8. The method of claim 7, wherein said cover part is secured to the annular trough by riveting, locking, adhering, or binding.

9. The method of claim 1. wherein said cover part completely or partially covers the injecting end of the said hollow fixation part.

10. The method of claim 1, wherein said cover part is an elastic cover part, a mesh-like cover part, a porous cover part, or a metal cover part.

11. The method of claim 1, wherein said cover part is cylindrical, cone-like, ball-like, quasi-ball, or quasi-cube in shape.

12. The method of claim 1, wherein said medical filling becomes is a cylindrical, cone-like, ball-like, quasi-ball, or quasi-cube lump after injecting is completed.

13. The method of claim 1, wherein the joining end of said hollow fixation part includes a tightening mechanism.

14. The method of claim 1, wherein said medical filling is a bone substitute, a bone cement, or a mixture or a composite thereof.

15. The method of claim 14, wherein said medical filling is a SrHA type of medical filling.

16. The method of claim 1, wherein said hollow fixation part is combination of multiple sections.
